SEQUENCE LISTING (24 sequences)

SEQ ID NO: 1 - protein, 805 aa, Homo sapiens [sequence omitted]

SEQ ID NO: 2 - protein, 726 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLG

SEQ ID NO: 3 - protein, 734 aa, Artificial Sequence
  ACE2 soluble ectodomain with heterologous (mouse IgG) 19 amino acid N-terminal signal peptide and 6x his tag amino acids 18-726 of precursor
MEWSWVFLFFLSVTTGVHSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGHHHHHH

SEQ ID NO: 4 - protein, 740 aa, Artificial Sequence
  ACE 2 soluble ectodomain with heterologous (mouse IgG) 19 amino acid N-terminal signal peptide (amino acids 18-732 of precursor), followed by 6xHis
MEWSWVFLFFLSVTTGVHSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGHHHHHH

SEQ ID NO: 5 - protein, 721 aa, Artificial Sequence
  ACE 2 soluble ectodomain (amino acids 18-732 of precursor) with 6xHis tag
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGHHHHHH

SEQ ID NO: 6 - protein, 723 aa, Artificial Sequence
  ACE2 soluble ectodomain (amino acids 18-740)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 7 - protein, 715 aa, Artificial Sequence
  ACE2 soluble ectodomain (amino acids 18-732)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLG

SEQ ID NO: 8 - protein, 709 aa, Artificial Sequence
  ACE2 soluble ectodomain (amino acids 18-726)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLG

SEQ ID NO: 9 - protein, 691 aa, Artificial Sequence
  ACE2 soluble ectodomain (amino acids 18-708)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSR

SEQ ID NO: 10 - protein, 673 aa, Homo sapiens
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNV

SEQ ID NO: 11 - protein, 598 aa, Artificial Sequence
  ACE2 minimal soluble ectodomain (amino acids 18-615)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 12 - protein, 715 aa, Artificial Sequence
  ACE2 soluble ectodomain (amino acids 18-732), R273K mutation
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGKFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLG

SEQ ID NO: 13 - protein, 740 aa, Artificial Sequence
  ACE2 soluble ectodomain (amino acids 18-732), R273K mutation, includes N-terminal heterologous signal peptide, C-terminal 6xhis tag
MEWSWVFLFFLSVTTGVHSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGKFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGHHHHHH

SEQ ID NO: 14 - protein, 721 aa, Artificial Sequence
  ACE2 soluble ectodomain (amino acids 18-732), R273K mutation, includes C-terminal 6xhis tag
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGKFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGHHHHHH

SEQ ID NO: 15 - protein, 709 aa, Artificial Sequence
  ACE2 soluble ectodomain (amino acids 18-726), R273K mutation
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGKFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLG

SEQ ID NO: 16 - protein, 734 aa, Artificial Sequence
  ACE2 soluble ectodomain (amino acids 18-726), R273K mutation, includes N-terminal heterologous signal peptide, C-terminal 6xhis tag
MEWSWVFLFFLSVTTGVHSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGKFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGHHHHHH

SEQ ID NO: 17 - protein, 715 aa, Artificial Sequence
  ACE2 soluble ectodomain (amino acids 18-726), R273K mutation, includes C-terminal 6xhis tag
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGKFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGHHHHHH

SEQ ID NO: 18 - DNA, 2223 bp, Artificial Sequence
  Encodes precursor of sACE2(R273K)6xhis (SEQ ID NO13)
atggagtggagctgggttttcctgttttttctgtccgtaaccacaggggtgcatagtcagtccactatcgaggagcaagctaaaactttcctggacaagtttaatcatgaggccgaggacctgttttaccagtcctcacttgcttcctggaattataatactaatatcactgaggaaaacgtgcaaaatatgaacaacgctggcgataaatggtctgctttcctgaaagagcagtcaacccttgctcagatgtatccacttcaggaaattcagaatctgactgttaaactgcagttgcaggccttgcagcagaacggatctagtgttctttctgaggataagtctaagaggctgaataccatcctgaatacaatgtcaaccatctacagtaccggaaaagtgtgtaacccagacaatcctcaggaatgcctcctgttggagccaggtcttaatgagattatggccaatagccttgactataacgaacgtctgtgggcatgggagtcatggcggtctgaagttggtaaacagctccgaccactgtacgaggagtacgttgtgctgaaaaatgagatggccagggccaatcattatgaagactacggagattactggagaggagattacgaagtcaacggggtcgacggttacgattacagtcggggtcaactgatcgaagacgtggagcatacctttgaggagataaagccactgtatgagcatcttcacgcatacgtgcgagctaagctgatgaatgcatatccaagttacatttcccctatcggttgtttgcctgcacatctgctcggtgatatgtgggggaagttctggaccaatctgtattccctgactgtgcccttcggccagaaaccaaacatcgatgttacagatgctatggtagatcaagcctgggatgctcagaggatttttaaggaggctgagaaattcttcgttagtgtgggcctccctaacatgacccagggtttttgggagaacagcatgctcactgaccccggcaatgtccagaaggccgtttgtcatccaaccgcctgggacttgggtaagggcgacttccgcatactgatgtgtaccaaagtcactatggatgacttcctcactgctcaccatgaaatgggacacattcagtatgacatggcctacgccgcccagccttttttgcttcgcaatggtgccaatgagggttttcacgaggccgtaggagagatcatgagcctgtctgccgccaccccaaaacatctcaaaagtatcggcctgctgtctccagatttccaggaagataatgaaaccgagattaacttcctgctcaaacaagcccttacaatcgtgggtaccttgcccttcacatatatgttggaaaaatggaggtggatggtgttcaaaggtgaaatccctaaggaccagtggatgaagaagtggtgggaaatgaagcgcgaaattgttggagtcgtggagccagtgcctcacgacgaaacttattgcgatcccgcctctctgtttcatgtgagcaacgactacagcttcattcgctactacacacggactctctatcagtttcagtttcaggaggccctgtgccaagctgctaaacacgaaggacctctgcacaaatgtgatattagcaactccaccgaggctggtcaaaagctctttaatatgctccgtctgggaaaatctgaaccttggactctggcacttgaaaatgtcgtgggggcaaagaatatgaatgtccgtccactcctcaactacttcgagcccctcttcacctggctgaaggatcagaacaagaactctttcgtcggatggtcaacagactggtcaccctatgctgatcagtcaattaaggtgcggatctccctcaaaagcgcactgggcgataaggcttacgagtggaacgataatgagatgtacttgtttaggtcttccgtggcctatgctatgaggcaatatttcctgaaagtgaaaaatcagatgatcctgtttggagaggaggacgtcagggtggcaaatctgaagcctagaatatcatttaacttctttgttactgcacctaagaacgtatctgatattattcccagaacagaggtggagaaggcaattcggatgagtagaagtcggattaacgatgctttcagactgaacgacaattctctggagttccttggaatccaacctacactgggccaccatcaccaccatcattga

SEQ ID NO: 19 - protein, 17 aa, Homo sapiens
MSSSSWLLLSLVAVTAA

SEQ ID NO: 20 - protein, 19 aa, Mus musculus
MEWSWVFLFFLSVTTGVHS

SEQ ID NO: 21 - protein, 29 aa, Homo sapiens
MNSFSTSAFGPVAFSLGLLLVLPAAFPAP

SEQ ID NO: 22 - protein, 6 aa, Artificial Sequence
  6x his tag
HHHHHH

SEQ ID NO: 23 - protein, 8 aa, Artificial Sequence
  8x his tag
HHHHHHHH

SEQ ID NO: 24 - protein, 10 aa, Artificial Sequence
  10x his tag
HHHHHHHHHH

(19) United States
(12) Patent Application Publication
(10) Pub. No.: US 2023/0158125 A1
(43) Pub. Date: May 25, 2023

(54) Pulmonary Administration of ACE2 Polypeptides

(71) Applicant: Sorrento Therapeutics, Inc., San Diego, CA (US)

(72) Inventors: Yanwen Fu, San Diego, CA (US); Yanliang Zhang, San Diego, CA (US); Heyue Zhou, San Diego, CA (US); Namir Shaabani, San Diego, CA (US); Reyna Lim, San Diego, CA (US); Yan Li, San Diego, CA (US); Chin-I Pai, San Diego, CA (US)

(73) Assignee: Sorrento Therapeutics, Inc., San Diego, CA (US)

(21) Appl. No.: 17/919,992

(22) PCT Filed: Apr. 20, 2021
(86) PCT No.: PCT/US2021/028155

§ 371 Date: Oct. 19, 2022

Related U.S. Application Data
(60) Provisional application No. 63/012,720, filed on Apr. 20, 2020.

(51) Int. Cl. A61K 38/48; A61K 9/00; A61P 31/14

(52) U.S. Cl. CPC A61K 38/4886; A61K 9/0075; A61P 31/14

(57) ABSTRACT

The present disclosure provides methods for treating a subject having a coronavirus infection by administering a composition that includes a sACE2 polypeptide to the lungs of a subject infected with a coronavirus. The sACE2 polypeptide includes the extracellular portion of the human ACE2 polypeptide and acts as a decoy, binding the spike (S) protein of coronavirus and thereby preventing the interaction of the S protein with membrane-associated ACE2 expressed on pulmonary cells, thus disrupting the infection process. The sACE2 polypeptide is derived from human ACE2, preventing potential immune reactions of the subject to the therapeutic polypeptide. The sACE2 polypeptide is administered locally to the site of the pathology, avoiding potential adverse effects of systemic delivery.

### CROSS REFERENCE TO RELATED APPLICATIONS

This application claims the benefit of priority to U.S. provisional application No. 63/012,720, filed Apr. 20, 2020, the content of which is incorporated herein by reference in its entirety.

### TECHNICAL FIELD

The invention relates to treating and preventing coronavirus infections by pulmonary delivery of protein-based therapeutic formulations.

### BACKGROUND

Coronavirus SARS-CoV-2 is the causative agent of the deadly Covid-19 pandemic. As is the case for the previously identified coronaviruses SARS-CoV and hCoV-NL63, SARS-CoV-2 infects human host cells using angiotensin converting enzyme II (ACE2) as a receptor (Walls et al. Cell 2020; doi.org/10.10.16/j.cell.2020.02.058). The ACE2 protein (precursor amino acid sequence Genbank NP 068576.1) is expressed on the surface of cells of the lung as well on cells of the heart, arteries, kidney, testes, liver, and intestines (Kuba et al. (2010) Pharmacol. Ther. 128:119-128). ACE2 is a single-pass Type I membrane protein having a peptidase domain in the extracellular region (ectodomain) of the protein. ACE2 hydrolyzes angiotensin II, a major effector peptide in the regulation of vasoconstriction and inflammation, among other physiological processes.

One factor contributing to the rapid spread of SARS-CoV-2 may be the affinity of the SARS-CoV-2 spike protein (S-protein, GenBank QHU36824) for the human ACE2 protein. Wang et al. (Cell (2020) doi.org/10.1016/j.cell.2020.03.045) determined that the C-terminal binding domain of the S protein of SARS-CoV-2 bound a sACE2 protein with approximately four times the affinity of the binding of the C-terminal binding domain of the S protein of SARS-CoV, the coronavirus responsible for the 2002-2003 SARS outbreak, while Wrapp et al. (2020; Science 367-1260-1263) estimated the binding of the ectodomain of ACE2 (the N-terminus up to amino acid 740 of the mature protein) to the S protein of SARS-CoV-2 to be ten to twenty-fold higher than the binding of the ACE2 ectodomain to the S protein of SARS-CoV. Lei et al. (2020, bioRxiv 2020.02.01.929976; doi:https://doi.org/10.1101/2020.02.01. 929976) disclosed that a fusion protein that included the ectodomain of the ACE2 protein fused to an antibody Fc region was able to prevent the binding of the SARS-CoV-2 S protein to the ACE2 polypeptide on engineered host cells in vitro.

A soluble recombinant form of human ACE2 (hrACE2) was administered intravenously in trials for treatment of acute respiratory distress syndrome (ARDS) (Kahn et al. (2017) Critical Care 21:234-42. Infusion of the hrACE2 was found to be well-tolerated but did not result in improvement in physiological or clinical measures of ARDS.

There is an urgent need to find therapeutics and prophylactic agents that can be rapidly deployed to halt the destructive spread of the SARS-CoV2 pandemic.

### SUMMARY

Provided herein are methods for treating a subject infected by a coronavirus, where the methods comprise administering an effective amount of a composition that includes a soluble ACE2 (sACE2) polypeptide to the respiratory tract, e.g., the lungs, of a subject infected with a coronavirus. The coronavirus can be any coronavirus that binds the human ACE2 receptor, such as, for example, the hCov-NL63 coronavirus, the SARS-CoV coronavirus, or the SARS-CoV-2 coronavirus. In some embodiments, the methods include administering an effective amount of a composition including a sACE2 polypeptide to the respiratory tract of a subject infected with the SARS-CoV-2 coronavirus. In preferred embodiments, the subject is a human subject and the composition is a pharmaceutical composition that includes a sACE2 polypeptide as disclosed herein and at least one pharmaceutically acceptable excipient, where the pharmaceutical composition is formulated for pulmonary delivery, such as by inhalation. One or more additional therapeutic compounds, such as for example, one or more antibiotics, one or more analgesics, or one or more anti-inflammatory compounds can optionally be administered with the sACE2 polypeptide. The administration of the composition can be to the lungs of the subject using any suitable means of inhalation delivery, including, without limitation, nebulizers and inhalers. Further provided are methods of preventing a coronavirus infection comprising administering an effective amount composition that includes a sACE2 polypeptide to the respiratory tract of a subject at risk of becoming infected with a coronavirus. The coronavirus can be any coronavirus that binds the human ACE2 receptor such as, for example, the hCov-NL63 coronavirus, the SARS-CoV coronavirus, or the SARS-CoV-2 coronavirus. In some embodiments, the methods are methods of preventing a SARS-CoV-2 infection, in which the methods include administering an effective amount of a composition that includes a sACE2 polypeptide to the respiratory tract, e.g, the lungs, of a subject at risk of becoming infected with the SARS-CoV-2 coronavirus. In preferred embodiments, the subject is a human subject and the composition is a pharmaceutical composition that includes a sACE2 polypeptide and at least one pharmaceutically acceptable excipient, where the pharmaceutical composition is formulated for pulmonary delivery, such as by inhalation. The administration of the composition can be to the lungs of the subject by inhalation using any suitable means of inhalation delivery, including, without limitation, nebulizers and inhalers.

In the methods provided herein, the sACE2 polypeptide composition is delivered to the lungs by inhalation, where the sACE2 polypeptide is provided in a formulation that can be aerosolized by a device such as a nebulizer or inhaler. The formulation can be a solid composition, e.g., a powder, or can be a liquid formulation, which can be a solution or suspension. Inhalers include, without limitation, metered dose inhalers (MDIs) and dry powder inhalers (DPIs). Nebulizers include, without limitation, piezoelectric sonication nebulizers, vibrating mesh nebulizers, and surface acoustic wave (SAW) nebulizers.

The sACE2 polypeptide of the composition comprises at least the amino acid sequence of SEQ ID NO:11 or sequence having at least 95% identity thereto, and preferably includes at least the S protein binding region of the soluble ectodomain of the human ACE2 protein (SEQ ID NO:10) or sequence having at least 95% identity thereto. In various embodiments the sACE2 polypeptide of the composition provided herein comprises an amino acid sequence having at least 95%, at least 96%, at least 97%, at least 98%, or at least 99% identity to SEQ ID NO:6, SEQ ID NO:7, SEQ ID NO:8, SEQ ID NO:9, or SEQ ID NO:10. The sACE2 polypeptide can include a peptide tag, such as a his tag, for example, can have 4, 6, 8, or 10 histidine residues at the N terminus of the polypeptide.

In various examples the sACE2 polypeptide used in the methods may be a mutant form of sACE2 that lacks peptidase activity. For example, the sACE2 polypeptide can include one or more mutations at positions R273, H345, H374, H378, and H505. For example, the sACE2 polypeptide can include one or more of the mutations R273K, H345A, H345L, H374N, H378N, H505A, and H505L. In exemplary embodiments the sACE2 comprises a mutation at R273, for example, comprises the R273K mutation. In some embodiments the sACE2 having the R273K mutation binds the S1 protein of SARS-CoV-2, and in some embodiments the sACE2 having the R273K mutation binds the RBD of the S1 protein of SARS-CoV-2. In various embodiments the sACE2 polypeptide having the R273K mutation binds to the S1 protein of SARS-CoV-2 with a KD that is substantially similar to the Kd for binding of an sADC2 polypeptide having the same amino acid sequence but lacking the R273K mutation. In some embodiments the sACE2 polypeptide does not include one or more mutations that enhance binding of the sACE2 polypeptide to the S1 protein or RBD of SARS-CoV-2. In particular embodiments the sACE2 polypeptide can include the R273K mutation. In some exemplary embodiments a sACE2 polypeptide of the compositions and methods disclosed herein comprises the amino acid sequence of SEQ ID NO:12, SEQ ID NO:14, SEQ ID NO:15, or SEQ ID NO:17.

Further included herein is a sACE2 polypeptide having a mutation at the R273 amino acid position, using the amino acid numbering according to the wild type precursor sequence (SEQ ID NO:1), where the mutant sACE2 polypeptide has reduced peptidase activity or substantially lacks peptidase activity. In some embodiments, a mutant sACE2 polypeptide of the compositions and methods disclosed herein comprises any of SEQ ID NOs:12 or 15, or a variant thereof, for example, a variant having at least 95%, at least 96%, at least 97%, at least 98%, or at least 99% identity to SEQ ID NO:12 or SEQ ID NO:17. The sACE2 polypeptide of the composition can further comprise additional amino acid sequences, such as a purification or detection tag, e.g., a carboxy-terminal tag of 4, 5, 6, 7, 8, 9, or 10 histidines, e.g., SEQ ID NO:14 or SEQ ID NO:17. In various embodiments the sACE2 polypeptide having the R273K mutation binds to the S1 protein of SARS-CoV-2 with a K_{D} that is similar to the K_{D} for binding of an sADC2 polypeptide having the same amino acid sequence but lacking the R273K mutation. In some embodiments the sACE2 polypeptide does not include one or more mutations that enhance binding of the sACE2 polypeptide to the S1 protein or RBD of SARS-CoV-2. In particular embodiments the sACE2 polypeptide can include the R273K mutation. In some exemplary embodiments a sACE2 polypeptide of the compositions and methods disclosed herein comprises the amino acid sequence of SEQ ID NO:12, SEQ ID NO:14, SEQ ID NO:15, or SEQ ID NO:17.

In addition to a sACE2 polypeptide, a composition for delivery to the lungs can include one or more carriers or excipients such as, for example, one or more buffering compounds, salts, metal ions, organic acids, amino acids, sugars, surfactants, stabilizers, alcohols, or polymers. The composition that includes a sACE2 polypeptide can be provided as a dry powder or as a liquid, which may optionally be frozen.

Further provided herein is a nucleic acid molecule encoding a mutant sACE, for example, a mutant sACE2 that comprises an R273K mutation. In various embodiments the encoded polypeptide is a precursor polypeptide that includes a signal peptide for secretion, e.g., a signal peptide of SEQ ID NO:19, SEQ ID NO:20, or SEQ ID NO:21. In some embodiments, the nucleic acid molecule encodes the sACE2 precursor polypeptide of SEQ ID NO:13 or SEQ ID NO:16.

### DESCRIPTION OF THE DRAWINGS

FIG. 1 is a schematic showing that a sACE2 polypeptide (pale green rectangles) that includes the extracellular domain of the ACE2 protein is able to bind the S protein on the coronavirus (circles). This binding of sACE2 polypeptide to S blocks the ACE2 binding site on the S protein, preventing the virus from binding to the human cell and causing infection. Where no sACE2 is present to block binding, the coronavirus (SARS-CoV-2) S spike protein (circles) engages cell-associated ACE2 (dark rectangles) which begins the infection process.

FIG. 2 is a schematic showing sACE2 polypeptide is delivered by inhalation to the lungs of the subject, providing several advantages: targeted and rapid delivery of the therapeutic agent to the site of infection; use of human protein that is not recognized as foreign by the subject's immune system; and the ease of combining the sACE2 polypeptide with other therapeutic agents.

FIG. 3 is a Surface Plasmon Resonance sensorgram of sACE2(R273K)-6his and sACE2-6his binding to the SARS-CoV2 S1 protein.

FIG. 4 is a Surface Plasmon Resonance sensorgram of sACE2(R273K)-6his and sACE2-6his binding to the RBD of the SARS-CoV2 spike protein RBD.

FIG. 5 provides the results of a FRNT assay of inhibition of infection of cultured cells by SARS-Cov-2.

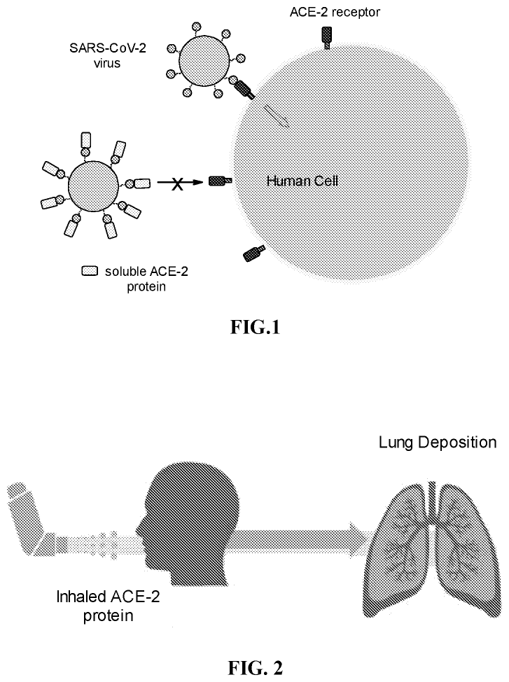

### DETAILED DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an”, and “the”, and as well as the use of the singular form of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “comprises”, “comprising”, “includes”, “including”, “having” and their conjugates mean “including but not limited to”. The term “comprising” also encompasses embodiments that “consist essentially of” and “consist of” the listed entity, quantity, or example(s).

The term “consisting essentially of” means that the composition, method or structure may include additional ingredients, steps and/or parts that do not materially alter the basic and novel characteristics of the claimed composition, method or structure.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. Alternatively, “about” or “comprising essentially of” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically synthesized forms. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Polypeptides such as sACE2 polypeptides can be prepared using recombinant procedures as described herein.

The terms “nucleic acid”, “polynucleotide” and “oligonucleotide” and other related terms used herein are used interchangeably and refer to polymers of nucleotides and are not limited to any particular length. Nucleic acids include recombinant and chemically synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding a sACE2 polypeptide.

The term “mutation”, “modification”, or “variation”, or related terms, refers to a change in a nucleic acid sequence or amino acid sequence that differs from a reference nucleic acid sequence or a reference amino acid sequence, respectively. Examples of mutations includes a point mutation, insertion, deletion, amino acid substitution, inversion, rearrangement, splice, sequence fusion (e.g., gene fusion or RNA fusion), truncation, transversion, translocation, non-sense mutation, sequence repeat, single nucleotide polymorphism (SNP), or other genetic rearrangement.

The term “recover” or “recovery” or “recovering”, and other related terms, refers to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide sequence which mediates secretion of the expressed protein. The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. Irrespective of the method used to recover the protein, the protein can optionally be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiments to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrophotometry. In various embodiments, any of the recombinant sACE2 polypeptides provided herein are isolated.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)2 fragments, Fab′ fragments and Fab fragments. Antibodies include single domain antibodies, monovalent antibodies, single chain antibodies, single chain variable fragment (scFv), camelized antibodies, nanobodies, affibodies, disulfide-linked Fvs (sdFv), anti-idiotypic antibodies (anti-Id), minibodies. Antibodies include monoclonal and polyclonal populations. Recombinant SACE2 polypeptides comprising an antibody domain that binds a coronavirus spike protein (S-protein) are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant (Ku) of 10⁻⁵M or less, or 10⁻⁶ M or less, or 10⁻⁷ M or less, or 10⁻⁸M or less, or 10⁻⁹M or less, or 10⁻¹⁰ M or less. Recombinant sACE2 polypeptides that specifically bind a coronavirus spike protein (S-protein) are described herein.

In one embodiment, a dissociation constant (K_{D}) can be measured using a surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. In some examples polypeptide sequence variants may have at least 85%, at least 90%, or at least 95% amino acid sequence identity with the referenced nucleic acid molecule or polypeptide. In various embodiments a variant polypeptide may have additional sequences at the N-terminus or C-terminus with respect to the reference polypeptide, for example, may include a detection or purification tag at the N or C terminus, or may include a localization sequence such as a nuclear localization sequence or signal peptide, or may lack a localization sequence present in the reference sequence. Variant proteins may include for example, insertions of amino acid sequences such as protease cleavage sites or amino acid tags for detection or purification. Further, a variant can include for example from one to six additional amino acids with respect to the reference protein or lack from one to six amino acid present in the reference protein due to variation in cellular processing events or minor clipping or alterations occurring during isolation or purification.

Preferably a protein variant has substantially the same activity or function as the protein from which it is derived. For example, a variant sACE2 polypeptide having at least 95% amino acid identity to a disclosed sACE2 polypeptide can have substantially the same binding affinity for a coronavirus S protein as the referenced sACE2 polypeptide.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation.

Similarly, a variant polynucleotide comprises a nucleotide sequence with one or more nucleotides inserted into, deleted from and/or substituted into the nucleotide sequence relative to another polynucleotide sequence. Variant polynucleotides also include polynucleotides having codon variations (e.g., codon-optimized genes). Polynucleotide variants include fusion polynucleotides.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters.

For example, sACE2 polypeptides can be at least 95%, or at least 96% identical, or at least 97% identical, or at least 98% identical, or at least 99% identical, to any of the polypeptides of SEQ ID Nos:6-10. In some embodiments, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

A host cell for the production of a sACE2 polypeptide can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector that includes a nucleic acid sequence encoding a sACE2 polypeptide thereby generating a transfected/transformed host cell which is cultured under conditions suitable for expression of the sACE2 polypeptide by the transfected/transformed host cell, and optionally recovering the antibody from the transfected/transformed host cells (e.g., recovery from host cell lysate) or recovery from the culture medium. In various embodiments the expression vector includes a nucleic acid sequence encoding a sACE2 polypeptide that includes a signal peptide directing secretion of the sACE2 polypeptide into the culture medium. The signal peptide is typically cleaved upon secretion of the sACE2 polypeptide, such that the signal polypeptide is substantially absent from the polypeptide provided in the final composition. In one embodiment, production host cells comprise non-human mammalian cells including CHO, BHK, NS0, SP2/0, Vero, and YB2/0. In one embodiment, production host cells comprise human cells such as HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), Vero cells, human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a production host cell is a mammalian host cell, but is not a human host cell. Typically, a production host cell is a cultured cell that can be transformed or transfected with a polypeptide-encoding nucleic acid, which can then be expressed in the host cell. The term production host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

Polypeptides of the present disclosure (e.g., sACE2 polypeptides) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine (his) tag, a FLAG tag, a myc tag, an HA tag, and a GST tag. Appropriate cloning and expression vectors for use with bacterial, fungal, yeast, and mammalian cellular hosts can be found in Cloning Vectors: A Laboratory Manual (Elsevier, N.Y., 1985).

An expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent such as a virus). Suitable host cells include prokaryotes, yeast, mammalian cells, or bacterial cells.

Suitable bacteria include gram negative or gram-positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988).

Preferred host cells for the production of a sACE2 polypeptide include mammalian host cell lines include endothelial cells, COS-7 monkey kidney cells, Vero CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts. Any of the recombinant sACE2 polypeptides, or fragment thereof, can be expressed by transgenic host cells.

SACE2 polypeptides disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized using molecular cloning and gene synthesis methods as they are known in the art, such as for example using chemically synthesized primers and enzymatic (polymerase) amplification. Gene synthesis is commercially available (e.g., DNA 2.0, Blue Heron, Genewiz, GeneScript, Synbio Technologies, GeneArt, etc.). Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al. (2003) Proc. Natl. Acad. Sci. USA 100:438-42; Sinclair et al. (2002) Protein Expr. Purif 96-105; Connell N D. Curr. Opin. Biotechnol. 2001 12(5):446-9; Makrides et al. (1996) Microbiol. Rev. 60:512-38; and Sharp et al. (1991) Yeast 7:657-78.

Polypeptides described herein can also be produced at least in part by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

SACE2 polypeptides described herein can be purified by isolation/purification methods for proteins generally known in the field of protein chemistry. Non-limiting examples include chromatography, including affinity chromatography, adsorption chromatography, ion exchange chromatography, hydrophobic chromatography, normal phase chromatography, reversed-phase chromatography, or any combinations of these. Other isolation/purification methods that may be employed include extraction, recrystallization, salting out (e.g., with ammonium sulfate or sodium sulfate), centrifugation, gel filtration, gel permeation chromatography, electrophoresis, and countercurrent distribution. After purification, polypeptides may be exchanged into different buffers and/or concentrated by any of a variety of methods known to the art, including, but not limited to, ultrafiltration and dialysis.

The purified sACE2 polypeptides described herein are preferably at least 65% pure, at least 75% pure, at least 85% pure, more preferably at least 95% pure, and most preferably at least 98% pure. Regardless of the exact numerical value of the purity, the polypeptide is sufficiently pure for use as a pharmaceutical product. Any of the recombinant sACE2 polypeptides, or fragment thereof, described herein can be expressed by transgenic host cells and then purified to about 65-98% purity or high level of purity using any art-known method.

In certain embodiments, the polypeptides herein can further comprise post-translational modifications. An exemplary post-translational protein modification is glycosylation. Post-translational modifications may also include phosphorylation, acetylation, methylation, ADP-ribosylation, ubiquitination, carbonylation, sumoylation, biotinylation or addition of a polypeptide side chain or of a hydrophobic group. As a result, the modified polypeptides may contain non-amino acid elements, such as lipids, poly- or monosaccharide, and phosphates.

In one embodiment, a sACE2 polypeptide described herein can be modified by linking the polypeptide to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). The present disclosure provides therapeutic compositions comprising any of the recombinant sACE2 polypeptides, described herein in an admixture with a pharmaceutically acceptable carrier and/or excipient. Pharmaceutically acceptable excipients include for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, surfactants, emulsifiers, antioxidants and isotonifiers. Salts, amino acids, sugars, alcohols, peptides, and polymers such as polyethylene glycol or polysorbate can also be provided in a pharmaceutical composition. The concentration of the sACE2 polypeptide in the pharmaceutical composition can vary depending upon a number of factors, including the dosage of the drug to be administered, and the route of administration.

Any of the recombinant sACE2 polypeptides may be optionally administered as a pharmaceutically acceptable formulation and may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include magnesium, zinc, iron, and the like.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. The sACE2 polypeptide compositions disclosed herein are delivered by pulmonary administration, i.e., by inhalation, preferably by oral inhalation, as described herein, e.g., using inhalers or nebulizers. Administering can be performed, for example, once, a plurality of times, and/or over one or more extended periods.

Other compounds or formulations that may be administered to a subject receiving therapy with the sACE2 treatments disclosed herein can use any pulmonary administration or can use an alternative route of administration, such as intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can be performed, for example, once, a plurality of times, and/or over one or more extended periods.

The terms “effective amount”, “therapeutically effective amount” or “effective dose” or related terms may be used interchangeably and refer to an amount of protein (e.g., any of the sACE2 polypeptides) that when administered to a subject, is sufficient to effect a measurable improvement or prevention of a disease, disorder, or pathology associated with coronavirus infection, including infection with SARS-CoV-2 (Covid-19). Therapeutically effective amounts of polypeptides provided herein, when used alone or in combination with other therapeutic compounds, will vary depending upon the relative activity of the polypeptides and combinations (e.g., in inhibiting cell growth) and depending upon the subject and stage of the disease being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered to a patient at about 0.01 mg to about 500 mg per day, preferably 0.01 mg to about 200 mg per day, most preferably 0.05 mg to about 100 mg per day. The polypeptide may be administered daily (e.g., once, twice, three times, or four times daily) or less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

sACE2 Polypeptide

The present disclosure provides compositions for treating a subject having a coronavirus infection where the compositions can administered to the respiratory tract, e.g., the lungs, of a subject infected, or at risk of becoming infected with, a coronavirus. The sACE2 polypeptide includes at least a portion of the ectodomain (e.g., at least the amino acid sequence of SEQ ID NO:10) of the human ACE2 polypeptide, or an amino acid sequence having at least 95%, at least 96%, at least 97%, at least 98%, or at least 99% identity to at least a portion of the human ACE2 ectodomain. In various embodiments the sACE2 polypeptide can include the amino acid sequence of SEQ ID NO:6, SEQ ID NO:7, SEQ ID NO:8, SEQ ID NO:9, or SEQ ID NO:10, or can include sequences having at least 95%, at least 96%, at least 97%, at least 98%, or at least 99% identity to of SEQ ID NO:6, SEQ ID NO:7, SEQ ID NO:8, SEQ ID NO:9, or SEQ ID NO:10. Without limiting the compositions and methods to any particular mechanism, it is postulated that in various embodiments the delivered sACE2 polypeptide can act as a decoy, binding coronavirus in the lungs and thereby preventing the interaction of the coronavirus S protein with ACE2 expressed on membranes of cells in the lungs, thus disrupting the infection process. The sACE2 therapeutic or prophylactic polypeptides provided herein are derived from human ACE2, thus the potential for immune reactions of the patient to the therapeutic or prophylactic polypeptide is minimized. The sACE2 polypeptide is administered locally to the site of the pathology, allowing for greater concentration of the therapeutic polypeptide at the site of pathology, and avoiding potential adverse effects of systemic delivery. The delivery can be repeated, and the frequency of delivery can be adjusted to maintain an optimal and modulatable local concentration of inhibitory sACE2 in the lung.

Various embodiments of a soluble human ACE2 polypeptide that may be used in the compositions and methods provided herein include sACE2 polypeptides that include the amino acid sequence of SEQ ID NO:11, which includes amino acids making contact with the S protein (Wang et al. 2020) or an amino acid sequence having at least 95%, at least 96%, at least 97%, at least 98%, or at least 99% identity to SEQ ID NO:11, where the sACE2 polypeptide binds a coronavirus S protein, such as the SARS-CoV-2 S protein. Further embodiments of a soluble human ACE2 polypeptide that may be used in the compositions and methods provided herein include sACE2 polypeptides that include the amino acid sequence of SEQ ID NO:12, which includes contact-making amino acids of the SARS-CoV-2 S protein binding domain of ACE2 (Wang et al. 2020) or an amino acid sequence having at least 95%, at least 96%, at least 97%, at least 98%, or at least 99% identity to SEQ ID NO:12, where the sACE2 polypeptide binds a coronavirus S protein, such as the SARS-CoV-2 S protein. In various embodiments, the ssACE2 polypeptide can have at least 95%, at least 96%, or at least 97%, for example, at least 98% or at least 99% identity to SEQ ID NO:6, SEQ ID NO:7, or SEQ ID NO:8.

In some embodiments, a sACE2 polypeptide can have mutations at amino acid position 273 (according to numbering of ACE2 precursor sequence (SEQ ID NO:1), or can have mutations at one or both of the histidines at amino acid positions 374 and 378, where such mutations may substantially reduce or eliminate peptidase activity of the sACE2 polypeptide which is not required for S protein binding (Lei et al., 2020). As used herein, “substantially reduce” the [peptidase] activity is meant to reduce the activity by at least 80%, 90%, or 95% with respect to control (wild type) activity.

In particular embodiments, a sACE2 polypeptide comprises the amino acid sequence of SEQ ID NO:12 or SEQ ID NO:15, or a sequence having at least 95%, at least 96%, or at least 97% identity, for example, at least 98%, or at least 99% identity to SEQ ID NO:12 or SEQ ID NO:15 includes an R273 mutation, such as an R273K mutation. An sACE2 polypeptide having the R273K mutation can bind the 51 protein of SARS-CoV-2, and preferably binds the SARS-CoV-2 S1 protein with a K_{D} that is comparable to the K_{D} of an sACE2 polypeptide that lacks the R273 mutation but is otherwise identical to the mutant sACE2 polypeptide. For example, binding affinity preferably differs from wild type binding affinity by less than an order of magnitude or preferably by less than 5 fold, less than 4 fold, less than 3 fold, or less than 2 fold.

In various embodiments, the sACE2 polypeptide of the compositions and methods provided herein, includes an sACE2 polypeptide that comprises a mutation that reduces or eliminates peptidase activity, does not include a mutation that enhances binding the S1 protein of a coronavirus such as SARS-CoV-2. Without wishing to be bound by theory, it is envisioned that the effectiveness of a therapeutic or prophylactic sACE2 polypeptide that is identical or close to identical in structural features (e.g., amino acids) that affect binding to the S1 protein will be less affected by variant forms of the SARS-CoV-2 polypeptide that may arise during an epidemic or pandemic, as the sACE2 polypeptide will adhere as closely as possible to the structure and binding properties of the native ACE2 receptor that is the target of any coronavirus variants that pose a threat.

In some embodiments, the sACE2 polypeptide does not include an immunoglobulin Fc region. In some embodiments, the sACE2 polypeptide is not fuse to an immunoglobulin Fc region.

In some embodiments, a sACE2 polypeptide of a composition provided herein, such as a pharmaceutical composition for the treatment or prevention of infection by a coronavirus, can include an amino acid sequence having at least 95%, at least 96%, at least 97%, at least 98%, or at least 99% identity to any of SEQ ID NOs:6-10. In some embodiments, a pharmaceutical composition for the treatment or prevention of infection by a coronavirus can include an amino acid sequence having at least 98%, or at least 99% identity to any of SEQ ID NOs:6-10. In some embodiments, a sACE2 polypeptide can have a mutation at position R273, or can have a mutation at any of H345, H505, or mutations at one or both of the histidines at amino acid positions 374 and 378 of SEQ ID NO:1 (ACE2 precursor sequence), where mutation of the histidines to amino acids such as but not limited to asparagine can diminish peptidase activity of the sACE2 polypeptide. In some examples, a sACE2 polypeptide as provided herein can have a mutation such as R273K, H345A, H345L, H374N, H378N, H505A, and H505L.

In some embodiments, a pharmaceutical composition for the treatment or prevention of infection by a coronavirus can include a sACE2 polypeptide having an R273K mutation and can comprise the amino acid sequence of SEQ ID NO:12 or SEQ ID NO:15, or a polypeptide having at least 95%, at least 96%, at least 97%, or at least 98% or at least 99% identity thereto. In some embodiments the sACE2 polypeptide does not include one or more mutations that enhance binding of the sACE2 polypeptide to the S1 protein or RBD of SARS-CoV-2. In some exemplary embodiments the sACE2 polypeptide of the compositions and methods disclosed herein can have the amino acid sequence that corresponds to the wild type human ACE2 amino acid sequence with the exception of a mutation at any of R273, H345, H374, H378, and/or H505. In various embodiments the sACE2 polypeptide includes the R273K mutation and does not have substantially less affinity for the S1 polypeptide than a sACE2 polypeptide that does not include the R273K mutation but is otherwise identical to the mutant sACE2. The sACE2 polypeptide can include an C-terminal his tag, for example, of 6, 8, or 10 histidine residues. In some embodiments the sACE2 polypeptide comprises SEQ ID NO:12 or SEQ ID NO:14.

The soluble human ACE2 polypeptides provided herein can lack the N-terminal signal peptide (amino acids 1-17 of the precursor ACE2 polypeptide (SEQ ID NO:19)), or in some cases may retain at least a portion of the signal peptide of the precursor ACE2 polypeptide or may include a different signal peptide or portion thereof, such as but not limited to a signal peptide of SEQ ID NO:20 or SEQ ID NO:21, or another signal peptide known in the art that results in secretion of the synthesized polypeptide in cells used for producing the sACE2 polypeptide. Processing of a signal peptide may differ in different cells, or may be non-uniform, such that proteins secreted by cells used for production of the soluble human ACE2 polypeptide may differ in the N-terminal by addition or deletion one, two, or three amino acids with respect the polypeptides of SEQ ID NOs:6-10, 12, and 15. The composition and methods encompass variants of sACE2 polypeptide where the N-terminus of the polypeptide may include from one to three additional amino acids with respect to SEQ ID NOs:6-10, 12, and 15, or may lack from one to three additional amino acids with respect to SEQ ID NOs:6-10, 12, and 15.

A sACE2 polypeptide as provided herein is able to bind a coronavirus S protein, such as a SARS-CoV-2 S protein (GenBank MN908947.3) with a K_{D} of 10⁻⁶ M or less, or preferably 10⁻⁷ M or less. Preferably the K_{D} of binding of a sACE2 polypeptide as provided herein to a SARS-CoV-2 S protein is 100 nM or less or 50 nM or less, and may be 20 nM or less. Methods of determining the K_{D} of S protein-sACE2 binding are known in the art (e.g., Lei et al. (2020), Walls et al. (2020); Wang et al. (2020); Wrapp et al. (2020)).

In some embodiments, the ACE2 polypeptide of the composition provided herein comprises at least the soluble ectodomain of the human ACE2 protein (sACE2, SEQ ID NO:10) or an amino acid sequence having at least 95% identity thereto, such as an amino acid sequence having at least 96%, 97%, 98%, or 99% identity to SEQ ID NO:10. In some embodiments the sACE2 polypeptide used in the compositions and methods provided herein has one or both histidines at positions 374 and 378 or SEQ ID NO:1 mutated to another amino acid such as but not limited to asparagine. The sACE2 polypeptide of the compositions provided herein can further comprise additional amino acid sequences, such as but not limited to purification or detection tags. In some embodiments, the composition comprises a sACE2 with a his tag (e.g., SEQ ID NO:4, SEQ ID NOs:6-9). A his tag can include from four to ten histidines (e.g., can have six, eight, or ten histidine residues). The sACE2 polypeptide can be glycosylated. In some embodiments the sACE2 polypeptide used in the compositions and methods provided comprises SEQ ID NO:6, SEQ ID NO:7, SEQ ID NO:8, SEQ ID NO:9, or SEQ ID NO:10. In some embodiments the sACE2 polypeptide used in the compositions and methods provided comprises SEQ ID NO:6, SEQ ID NO:7, SEQ ID NO:8, SEQ ID NO:9, or SEQ ID NO:10. In some embodiments the sACE2 polypeptide used in the compositions and methods provided comprises SEQ ID NO:6. In some embodiments the sACE2 polypeptide used in the compositions and methods provided comprises a variant of SEQ ID NO:6 where amino acids 374 and 378 (numbering as in SEQ ID NO:1) are mutated from histidine to another amino acid such as asparagine.

In some embodiments a sACE2 polypeptide may be a precursor polypeptide, such as but not limited to any of SEQ ID NO:2, SEQ ID NO:3, SEQ ID NO:4, of SEQ ID NO:5, SEQ ID NO:6, or variants having at least 95% identity thereto or having other signal peptides as they are known in the art.

In addition to the sACE2 polypeptide, a composition for delivery to the lungs can be a pharmaceutical composition and can include one or more carriers or excipients such as, for example, one or more buffering compounds, salts, metal ions, organic acids, amino acids, sugars, surfactants, stabilizers, alcohols, or polymers. The pharmaceutical composition that includes a sACE2 polypeptide can be provided as a dry powder or as a liquid, which may be frozen.

### Dry Powder Formulations

In various embodiments, a sACE2 polypeptide is formulated in dry powder formulation suitable for inhalation. A dry powder formulation comprising the protein composition has particles of a size range compatible with the delivery device, which may be, e.g., a metered dose inhaler or dry powder inhaler. Dry powder formulations for inhalation therapy are known in the art and described in U.S. Pat. No. 5,993,805 to Sutton et al.; U.S. Pat. No. 6,921,527 to Platz et al.; WO 0000176 to Robinson et al.; WO 9916419 to Tarara et al.; WO 0000215 to Bot et al; U.S. Pat. No. 5,855,913 to Hanes et al.; and U.S. Pat. Nos. 6,136,295 and 5,874,064 to Edwards et al., all of which are incorporated by reference in their entireties.

A dry powder formulation comprising a sACE2 polypeptide can include, in addition to a sACE2 polypeptide as disclosed herein that comprises an amino acid sequence having at least 95% identity to any of SEQ ID NOs:1-4, one or more pharmaceutically acceptable carriers. For example, a dry powder sACE2 polypeptide composition may further comprise a bulking agent or stabilizer. Carrier molecules can include, without limitation, sugars, sugar alcohols, polyols, amino acids, and polymers. For examples, amino acids such as glycine, leucine, trileucine, alanine, valine, arginine, histidine, or methionine may be included in the formulation. Alternatively or in addition, sugars such as but not limited to mannose, sucrose, fructose, maltose, lactose, or trehalose may be included. Salts such as but not limited to magnesium, calcium, sodium, potassium, and lithium salts may be present in a dry powder formulation for pulmonary delivery. Sorbitol, mannitol, or other sugar alcohols or polymers such as polyethylene glycol, polysorbate, microcrystalline cellulose, or starch may also be present in a dry powder composition.

One or more additional active compounds can also be incorporated into a dry powder sACE2 polypeptide composition for pulmonary delivery. For example, a sACE2 polypeptide may be co-formulated with one or more additional compounds such as but not limited to one or more anti-inflammatory agents, analgesics, bronchodilators, or antibiotics, or any combination thereof.

Dry powder compositions may be formulated from liquid solutions of the sACE2 polypeptide that are lyophilized or spray dried; such solutions can include one or more buffering agents, stabilizers, or surfactants that may also be present in the dry powder formulation. For example, a buffering agent present in the formulation can include phosphate, citrate, succinate, histidine, imidazole, or Tris. EDTA may be present as a stabilizer. Any of various surfactants may also be present, such as for example, polyoxyethylene sorbitol esters such as polysorbate 80 (Tween 80) and polysorbate 20 (Tween 20); polyoxypropylene-polyoxyethylene esters such as Poloxamer 188; polyoxyethylene alcohols such as Brij 35; a mixture of polysorbate surfactants with phospholipids (such as phosphatidylcholine and derivatives), dimyristoylglycerol and other members of the phospholipid glycerol series; lysophosphatidylcholine and derivatives thereof; mixtures of polysorbates with lysolecithin or cholesterol; bile salts and their derivatives such as sodium cholate, sodium deoxycholate, sodium glycodeoxycholate, sodium taurocholate, etc. Examples of suitable surfactants include L-alpha-phosphatidylcholine dipalmitoyl (“DPPC”), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, and alkylated sugars. A surfactant such as but not limited to, sorbitan trioleate, soya lecithin, and oleic acid may be added to the formulation to reduce adhesion of the protein-containing dry powder to the walls of the delivery device from which the aerosol is dispensed.

A liquid formulation (such as but not limited to any described hereinbelow) comprising a sACE2 polypeptide as provided herein, e.g., a polypeptide comprising any of SEQ ID NOs:6-10 or a variant thereof, such as a variant including a polypeptide having a sequence having at least 95% identity to any of SEQ ID NOs:6-10, can be processed into a dry powder form using lyophilization or spray-drying techniques well known in the art. Powder comprising a sACE2 polypeptide as disclosed herein may also be prepared using methods known in the art, including crystallization or precipitation (see, for example, dry powder microspheres (PROMAXX; Baxter) described in U.S. Pat. Nos. 5,525,519; 5,599,719; 5,578,709; 5,554,730; 6,090,925; 5,981,719; 6,458,387, each of which is incorporated herein by reference).

For example, a liquid sACE2 composition, such as a liquid formulation as disclosed herein, can be lyophilized and the lyophilized composition can be milled to obtain the finely divided dry powder consisting of particles within the desired size range. Where spray-drying is used to obtain a dry powder form of a liquid composition, the process can be carried out under conditions that result in a substantially amorphous finely divided dry powder consisting of particles within the desired size range. Methods of preparing dry powder forms of formulations, are known in the art and disclosed, for example, in WO 96/32149, WO 97/41833, WO 98/29096, and U.S. Pat. Nos. 5,976,574, 5,985,248, and 6,001,336; all herein incorporated by reference.

In some embodiments, an inhalable dry powder formulation as provided herein comprises finely divided particles, coarse particles, or a combination of fine and coarse particles. “Finely divided particles” includes particles of generally 0.5 to 100 microns in diameter, which may be in the range of 1-50 microns mass median diameter (MMD), in the range of 1-25 microns mass median diameter (MMD), in the range of 1-6 microns mass median diameter (MMD), in the range of 0.1-0.5 microns mass median diameter (MMD), in the range of 0.5-1.0 microns mass median diameter (MMD), in the range of 1.0-2.0 microns mass median diameter (MMD), or in the range of 2.0-5 microns mass median diameter (MMD). As used herein, the term “coarse particles” includes particles of generally greater than 50 microns in diameter, in the range of 50-500 microns mass median diameter (MMD), or in the range of 150-400 microns MMD. The size ranges of particles will depend in part on the delivery device. Particles comprising a sACE2 polypeptide as provided herein should be of respirable size: that is, particles of a size sufficiently small to pass through the mouth and larynx upon inhalation and into the bronchi and alveoli of the lungs. Preferably, particles in a dry powder composition as provided herein range from about 0.2 to about 20 microns in size, more preferably from about 0.5 microns to about 10 microns in size, and in various preferred embodiments may be less than about 5 microns in size (See, e.g., U.S. Pat. No. 5,292,498 to Boucher, US20070044793 to Kleinstreuer et al. or US20110056492 to Longest et al.).

The resulting dry powder composition can be placed within an appropriate delivery device for delivery to the subject as an aerosol via pulmonary inhalation. Alternatively, the dry powder form of the formulation is prepared and dispensed as an aqueous or nonaqueous solution or suspension via a metered-dose inhaler or other appropriate delivery device that delivers an aerosol for pulmonary inhalation. The amount of dry powder form of the composition placed within the delivery device is sufficient to allow for delivery of a pharmaceutically effective amount of the composition to the subject by inhalation. For example, following placement of the dry powder form within a delivery device, the particles are suspended in an aerosol propellant. The pressurized nonaqueous suspension is released from the delivery device into the respiratory tract of the subject while inhaling.

The dry powder formulation of the invention can be formulated to comprise a dose that may be, for example, from about 1 μg (0.001 mg) to about 200 mg, or from about 10 μg (0.01 mg) to about 100 mg, or from about 50 μg to about 100 mg of the sACE2 polypeptide disclosed herein. Specific dosage regimens can be adjusted over time according to the condition and characteristics of the individual patient and the professional judgment of the person administering or supervising the administration of the compositions. Dosage ranges set forth herein are exemplary only and are not intended to limit the scope or practice of the claimed composition.

In some embodiments, the dry powder compositions of some embodiments may be provided in a pack or dispenser device, which may contain one or more unit dosage units, for example, the composition can be provided in a container that includes a metal or plastic foil, such as a blister pack containing individual dosages. The pack or dispenser device may be provided as a kit accompanied by instructions for administration. The pack or dispenser may also include labeling approved by the U.S. Food and Drug Administration for prescription drugs or of an approved product insert.

The pack or kit optionally can include a second agent (e.g, an analgesic, anti-inflammatory, anti-viral, or antibiotic) packaged with or copromoted with instructions for using the second agent with the sACE2 polypeptide formulation.

### Liquid Formulations

A sACE2 polypeptide, such as any disclosed herein, can be incorporated into a liquid pharmaceutical composition suitable for pulmonary administration to a subject. For example, a liquid pharmaceutical composition as provided herein can include a sACE2 polypeptide and a pharmaceutically acceptable carrier, where pharmaceutical composition is suitable for inhalation by a subject. Examples of pharmaceutical compositions which are suitable for inhalation include, but are not limited to, a propellant-containing aerosol and a propellant-free inhalable solution or suspension.

Provided herein is a liquid composition that comprises a sACE2 polypeptide as disclosed herein (e.g., a polypeptide comprising the sequence of any of SEQ ID NOS:6-10, or a variant thereof, including a variant comprising a sequence having at least 95% identity to any of SEQ ID NOs:6-10) is provided where the liquid composition is formulated for pulmonary administration. The sACE2 polypeptide composition is formulated into a solution or suspension, e.g., an isotonic saline solution, which is optionally buffered, at an appropriate concentration for pulmonary administration as an aerosol, mist, or vapor. Preferably, a solution or suspension that includes a sACE2 polypeptide is isotonic with respect to pulmonary fluids and of about the same pH, for example, has a pH of from about pH 4.0 to about pH 8.5 or from pH 5.5 to pH 7.8. For example, the pH can be from about 7.0 to about 8.2. Suitable buffering agents that can be present in a liquid pharmaceutical composition for pulmonary delivery include, but are not limited to, citrate buffer, phosphate buffer, and succinate buffer. Alternatively or in addition, imidazole, histidine, or another compound that maintains pH in the range of about pH 4.0 to about 8.5 can be used. For example, Ringer's solution, isotonic sodium chloride, and phosphate buffered saline may be used. One of skill in the art can determine an appropriate saline content and pH for an aqueous solution for pulmonary administration.

Additional compounds that may be present in a liquid formulation for pulmonary delivery include, without limitation, the compounds provided above for dry powder formulations, including: sugars, sugar alcohols, polyols, amino acids, and polymers; amino acids, salts, polymers, surfactants, and preservatives (e.g., ethyl or n-propyl p-hydroxybenzoate). Other possible ingredients include suspending agents such as cellulose derivatives, sodium alginate, polyvinyl-pyrrolidone, and gum tragacanth, and a wetting agent such as lecithin. The compositions can include any of a variety of compounds to aid in solubility, stability, or delivery, where the added compounds do not negatively affect the coronavirus S-protein binding activity of the sACE2 protein.

For example a liquid pharmaceutical composition for pulmonary delivery as provided herein may include an excipient or stabilizer including but not limited to a sugar, sugar alcohol, or an amino acid. Preferred sugars include sucrose, trehalose, raffinose, stachyose, sorbitol, glucose, lactose, dextrose, or any combination thereof. For example, a sugar, can be present in the range of about 0% to about 9.0% (w/v), preferably about 0.5% to about 5.0%, for example about 1.0%; an amino acid, can be present in the range of about 0% to about 1.0% (w/v), preferably about 0.3% to about 0.7%, for example about 0.5%.

Other ingredients as provided above for solid formulations may also be present, including an alcohol (e.g., benzyl alcohol), stabilizers such as EDTA, and one or more lipids or surfactants, for example, any of the lipids or surfactants disclosed hereinabove.

Such pharmaceutical compositions may be administered for example, as a propellant-free inhalable solution comprising a sACE2 polypeptide and may be administered to the subject via a nebulizer. Other suitable preparations include, but are not limited to, mist, vapor, or spray preparations so long as the particles comprising the protein composition are delivered in a size range consistent with that described for the delivery device.

Therapeutic compositions are preferably sterile and stable under the conditions of manufacture and storage. The formulation can be formulated as a solution, microemulsion, dispersion, or suspension. Sterile inhalable solutions can be prepared by incorporating the active compound (i.e., a sACE2 polypeptide as provided herein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. Fluidity of a solution can be maintained, for example, by the use of a coating such as lecithin, by the maintenance of the required particle size in the case of dispersion, and/or by the use of surfactants.

The concentration of sACE2 polypeptide in a liquid formulation for pulmonary delivery can range for example, from about 1 μg per ml to about 500 mg per ml, and may be in the range of, for example, from about 10 μg per ml to about 200 mg per ml, or from about 20 μg per ml to about 100 mg per ml, although these ranges are not limiting.

### Methods of Treatment

Provided herein are methods of treating a subject with a coronavirus infection or at risk of becoming infected with a coronavirus. The subject can be a human subject and can be a patient testing positive for a coronavirus such as hCov-NL63, SARS-CoV, or SARS-CoV-2. In some embodiments the subject is a subject testing positive for SARS-CoV-2, exhibiting symptoms of Covid-19, or at risk of being infected with SARS-CoV-2. The methods include treating a subject infected with a coronavirus or suspected of being infected with a coronavirus where the methods include administering a composition comprising a sACE2 polypeptide, such as any disclosed herein, to the respiratory tract of the subject. The methods further include preventing a subject at risk of becoming infected with a coronavirus from becoming infected with a coronavirus where the methods include administering a composition comprising a sACE2 polypeptide, such as any disclosed herein, to the respiratory tract of the subject. In various embodiments of treatment and prophylaxis the composition is administered by pulmonary delivery, for example by oral inhalation. Pulmonary delivery can use any delivery device that can deliver a liquid (e.g., droplets) or particulate composition to the lungs, e.g., can deliver aerosols comprising a therapeutic composition such as a liquid or dry powder pharmaceutical composition as provided herein to the lungs.

The pharmaceutical compositions provided herein include a sACE2 polypeptide that specifically binds a coronavirus S protein, such as a SARS-CoV-2 coronavirus S protein (GenBank QHU36824). The sACE2 polypeptide can be any as described hereinabove, and can be a sACE2 polypeptide comprising an amino acid sequence having at least 95% identity to any of SEQ ID NOs:6-10, for example, can comprise any of SEQ ID NOs:6-10.

Administration of a pharmaceutical formulation that include a sACE2 polypeptide is by inhalation for pulmonary delivery, and can use any device that provides respiratable droplets or particles that are able to reach the lungs by inhalation. For example, pulmonary delivery can be by means of a delivery device such as but not limited to a nebulizer, a metered dose inhaler, or a dry powder inhaler.

The formulations of the invention may include a “therapeutically effective amount” or a “prophylactically effective amount” of a sACE2 polypeptide as provided herein. A “therapeutically effective amount” refers to an amount effective, at dosages and for periods of time necessary, to achieve the desired therapeutic result. A therapeutically effective amount of the DNase may vary according to factors such as the viral load, disease state, age, sex, and weight of the individual, and the ability of the sACE2 polypeptide to elicit a desired response in the individual. A therapeutically effective amount is also one in which any toxic or detrimental effects of the sACE2 polypeptide are outweighed by the therapeutically beneficial effects. A “prophylactically effective amount” refers to an amount effective, at dosages and for periods of time necessary, to achieve the desired prophylactic result. Typically, although not necessarily, since a prophylactic dose is used in subjects prior to or at an earlier stage of disease, the prophylactically effective amount will be less than the therapeutically effective amount.

Toxicity and therapeutic efficacy of the active ingredients described herein can be determined using standard pharmaceutical procedures including in vitro, in cell cultures, and experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually, for example, to provide serum and cell levels of the active ingredient which are sufficient to induce or suppress the biological effect (minimal effective concentration, MEC). Dosages necessary to achieve the MEC will depend on individual characteristics including the severity of the viral infection and related pathologies, the condition of the patient, and the judgment of the physician. Depending on the severity and responsiveness of the condition to be treated, dosing can be of a single or a plurality of administrations, with course of treatment lasting from several days to several weeks or until cure is effected or diminution of the disease state is achieved.

The delivery device can deliver, in a single dose or in multiple doses, a pharmaceutically effective amount of the composition to the subject's lungs by pulmonary inhalation. Devices suitable for pulmonary delivery of a dry powder form of a protein composition as a nonaqueous suspension are commercially available. Examples of such devices include the Ventolin metered-dose inhaler (Glaxo Inc., Research Triangle Park, N.C.) and the Intal Inhaler (Fisons, Corp., Bedford, Mass.). See also the aerosol delivery devices described in U.S. Pat. Nos. 5,522,378, 5,775,320, 5,934,272 and 5,960,792, herein incorporated by reference. An aerosol propellant used in an aerosol delivery device may be any conventional material employed for this purpose, such as a chlorofluorocarbon, a hydrochloro-fluorocarbon, a hydrofluorocarbon, or a hydrocarbon, including trichlorofluoromethane, dichlorodifluoro-methane, dichlorotetrafluoromethane, dichlorodifluoro-methane, dichlorotetrafluoroethanol, and 1,1,1,2-tetra-fluoroethane, or combinations thereof.

Where the solid or dry powder form of the formulation is to be delivered as a dry powder form, a dry powder inhaler or other appropriate delivery device is preferably used. The dry powder form of the formulation is preferably prepared as a dry powder aerosol by dispersion in a flowing air or other physiologically acceptable gas stream. For example, the delivery device can be any of dispenser is of a type selected from the group consisting of a reservoir dry powder inhaler (RDPI), a multi-dose dry powder inhaler (MDPI), and a metered dose inhaler (MDI).

A reservoir dry powder inhaler (RDPI) is an inhaler having a reservoir form pack suitable for comprising multiple (un-metered) doses of a formulation in dry powder form and can include means for metering medicament dose from the reservoir to a delivery position. The metering means may for example comprise a metering cup, which is movable from a first position where the cup may be filled with the composition to be delivered from the reservoir to a second position where the metered formulation dose is made available to the subject for inhalation.

A multi-dose dry powder inhaler (MDPI) is an inhaler suitable for dispensing a pharmaceutical formulation in dry powder form, wherein the pharmaceutical formulation is within a multi-dose pack containing multiple, defined doses of the pharmaceutical formulation. In one embodiment, the carrier has a blister pack form.

In the case of multi-dose delivery, the pharmaceutical formulation can be pre-metered (e.g., as in Diskus, see GB 2242134, U.S. Pat. Nos. 6,632,666, 5,860,419, 5,873,360 and 5,590,645, or Diskhaler, see GB 2178965, 2129691 and 2169265, U.S. Pat. Nos. 4,778,054, 4,811,731, and 5,035,237, the disclosures of each of which are hereby incorporated by reference) or metered in use (e.g., as in Turbuhaler, see EP 69715 or in the devices described in U.S. Pat. No. 6,321,747, the disclosures of each of which are hereby incorporated by reference). An example of a unit-dose device is Rotahaler (see GB 2064336 and U.S. Pat. No. 4,353,656, the disclosures of each of which are hereby incorporated by reference).

A metered dose inhaler (MDI) is a pharmaceutical formulation dispenser suitable for dispensing the formulation in aerosol form, where the aerosol container includes a propellant-based aerosol containing the pharmaceutical formulation. The aerosol container is typically provided with a metering valve, for example a slide valve, for release of the aerosol formulation to the subject. The aerosol container is generally designed to deliver a predetermined dose of the therapeutic formulation upon each actuation by means of the valve, which can be opened either by depressing the valve while the container is held stationary or by depressing the container while the valve is held stationary.

Spray compositions for topical delivery to the lung by inhalation may for example be formulated as suspensions or as aerosols delivered from pressurized packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetrafluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurized formulations will generally be retained in a canister (e.g., an aluminum canister) closed with a valve (e.g., a metering valve) and fitted into an actuator provided with a mouthpiece.

Liquid aerosol delivery is another form of pulmonary drug delivery that can be employed. Liquid aerosols are created by nebulizers, which releases compressed air from a small orifice at high velocity, resulting in low pressure at the exit region due to the Bernoulli effect. See, e.g., U.S. Pat. No. 5,511,726. The low pressure is used to draw the fluid to be aerosolized out of a second tube. This fluid breaks into small droplets as it accelerates in the air stream. Nebulizers are generally more effective for deliver to the deep lung. Type of nebulizers for liquid formulation aerosolization include, for example, air jet nebulizers, liquid jet nebulizers, ultrasonic nebulizers, and vibrating mesh nebulizers. Examples of nebulizers include the Akita™ (Activaero GmbH) (see U.S. Pat. No. 7,766,012, EP1258264 and the portable Aeroneb™ Go, Pro, and Lab nebulizers (AeroGen). A dry powder formulation can also delivered by a nebulizer. The nebulizer can use any pharmaceutically acceptable carrier, including a saline solution. Nebulizers can be customized for delivery of the particular protein, e.g. a sACE2 polypeptide, to reduce any denaturation, aggregation, and loss of activity during nebulization.

Ultrasonic nebulizers use flat or concave piezoelectric disks submerged below a liquid reservoir to resonate the surface of the liquid reservoir, forming a liquid cone which sheds aerosol particles from its surface (U.S. 2006/0249144 and U.S. Pat. No. 5,551,416). Since no airflow is required in the aerosolization process, high aerosol concentrations can be achieved. Smaller and more uniform liquid respirable dry particles can be obtained by passing the liquid to be aerosolized through micron-sized holes. See, e.g., U.S. Pat. Nos. 6,131,570; 5,724,957; and 6,098,620.

Vibrating mesh nebulizers, which are considered less likely to cause protein denaturation (Bodier-Montagutelli et al. (2018) Exp Op Drug Deliv 15:729-736), may be used to generate aerosols for delivery of a liquid composition the includes a sACE2 polypeptide to the lungs of a subject. Vibrating mesh nebulizers force liquid through a vibrating membrane with apertures of specific sizes, resulting in droplets having diameters within a specified range. Examples of vibrating mesh nebulizers include the ALX-0171 Nanobody™ nebulizer, the Vectura FOS-Flamingo®, the PARI eFlow®, the Philips I-neb AAD®, and the Aeroneb™ Pro (Rohm et al. (2017) Intl J Pharmaceutics 532:537-546; Bodier-Montagutelli et al. (2018)).

In various embodiments, a dosage regimen can include a single dose of a dry powder or liquid formulation of the invention, of 0.001 to 500 mg sACE2 polypeptide, or about 10 μg to 200 mg sACE2 polypeptide, administered multiple times, for example, daily, every other day, or weekly, or a plurality of doses administered at least twice, 2-3 times, 2-4 times or 2-6 times daily; or a plurality of doses administered once every 36 hours, once every 36-48 hours, once every 36-72 hours, once every 2-3 days, once every 2-4 days, once every 2-5 days, or once every week; or a plurality of doses administered once every 36 hours, once every 36-48 hours, once every 36-72 hours, once every 2-3 days, once every 2-4 days, once every 2-5 days, or once every week. The dosage schedule can be modulated to accommodate the condition of the patient, including but not limited to viral load, antibody response, and respiratory symptoms.

In some embodiments, the method further comprises detecting reduced infection or reduced viral load of the coronavirus in a subject diagnosed as having a coronavirus infection after pulmonary delivery of a sACE2 polypeptide as provided herein to the subject with a soluble ACE2 polypeptide, compared to infection of the target cell by the coronavirus in the absence of the recombinant ACE2 fusion polypeptide.

The disclosure provides methods for treating or preventing coronavirus infection by delivering to the lungs of a subject having or suspected of having a coronavirus infection a composition that includes a polypeptide comprising a sACE2 polypeptide formulated for pulmonary administration. The composition can be a pharmaceutical composition that includes, in addition to a polypeptide that comprises an ACE2 ectodomain, at least one pharmaceutically acceptable excipient or carrier compound. The pharmaceutical formulation that includes a sACE2 polypeptide can be a formulation for delivery by aerosol inhalation and can be in solid or liquid form. Compositions as provided herein can be packaged in single dose units, for example, in blister packs, vials, or dispensers such as, for example dry powder inhalers, metered-dose inhalers, or nebulizers that generate aerosols for delivery of particles or droplets to the lung.

The sACE2 polypeptide used in the formulations provided herein binds to a coronavirus, such SARS-CoV, SARS-CoV-2, or hCov-NL63 with high affinity. The sACE2 polypeptide binds to the S-protein of a coronavirus, such as the SARS-CoC-2 S protein. The sACE2 polypeptides described herein block binding between coronavirus S-protein (e.g., the SARS-CoC-2 S protein) and ACE2 receptor on cells of the lung, thereby reducing or preventing coronavirus infection. In some preferred embodiments, the polypeptide that comprises an ACE2 domain does not include a transmembrane domain (amino acids 741-761 of SEQ ID NO:1). In some embodiments, the polypeptide that includes an ACE2 ectodomain (SEQ ID NO:10) or consists essentially of an ACE2 ectodomain.

Further provided herein are methods of delivering a sACE2 polypeptides, such as any disclosed herein, formulated for pulmonary administration to the lungs of a subject, such as a human subject, by inhalation. The pharmaceutical formulations that include a sACE2 polypeptide can be formulations for delivery by aerosol inhalation and can be in solid or liquid form. The compositions can be packaged in single dose units, for example, in blister packs, vials, or dispensers such as, for example dry powder inhalers, metered-dose inhalers, or nebulizers that generate aerosols for delivery of particles or droplets to the lung.

In one embodiment, the recombinant sACE2 polypeptide provided in a pharmaceutical composition as described herein blocks binding between a coronavirus and an ACE2 receptor on a target cell, by binding to a the S protein) on a SARS-Cov-2 virus (GenBank MN908947.3) or a SARS-CoV virus.

In one embodiment, the recombinant soluble ACE polypeptides described herein reduces entry of a coronavirus, for example, a SARS-CoV-2 virus, into a target cell expressing an ACE2 receptor, by binding to the S protein on a SARS-CoV-2 virus (GenBank MN908947.3) or a SARS virus.

In one embodiment, a sACE2 polypeptide of a composition provided herein comprises the ectodomain of human ACE2 (SEQ ID NO:10) or comprises an amino acid sequence having at least 95% identity thereto, where the sACE2 polypeptide binds the SARS-CoV-2 S protein (GenBank MN908947.3) with high affinity, e.g., with a K_{D} of 10⁻⁶ M or less, or preferably 10⁻⁷ M or less.

In various embodiments the sACE2 polypeptide comprises SEQ ID NO:6, SEQ ID NO:7, SEQ ID NO:8, SEQ ID NO:9, or SEQ ID NO:10. In some embodiments, the ACE2 polypeptide comprises an affinity purification tag (e.g., His-tag) at the N-terminus or C-terminus.

### EXAMPLES

### Example 1. Soluble ACE2 Protein Construct sACE2(R273K)-6his

A DNA construct that encoded a soluble ACE2 polypeptide that included the ectodomain of human ACE2 protein having the R273K mutation (sACE2(R273K) and a C-terminal 6 histidine tag and having an N-terminal signal peptide (SEQ ID NO:13) for secretion was produced by recombinant methods and cloned into a mammalian expression vector for production of the sACE2(R272K) protein in CHO (chinses hamster ovarian cells) cells. The sACE2(R273K) protein in was purified from media harvested from the CHO cell culture on a column that included nickel-charged nitrilotriacetic acid (NTA) coupled to a cross-linked 6% agarose resin) using standard IMAC (Immobilized Metal Affinity Chromatography) methods.

For use as controls, a soluble ACE2 polypeptide having the ectodomain of the wild type ACE2 polypeptide, and a C-terminal 6×his tag (SEQ ID NO:5), was produced the same way from a construct encoding the precursor having an N-terminal signal peptide (SEQ ID NO:4). Experiments using the sACE2(R273K) polypeptide (SEQ ID NO:12) fused to a human IgG Fc region demonstrated that the soluble ACE2 polypeptide having the R273K mutation in fact lacked the peptidase activity observed in the wild type ACE2 soluble domain in accordance with the results of Guy et al. (2005) FEBS J. 272:3512-3520.

### Example 2. Binding Affinity of sACE2 to the RBD of the SARS-CoV-2 S Protein

Binding kinetics of sACE2(R273K)-6his (SEQ ID NO:12) and the sACE2-6his polypeptide that lacked the R273K mutation (SEQ ID NO:5) with the SARS-CoV-2 S protein and the RBD of the SARS-CoV-2 S1 protein were measured using surface plasmon resonance (SPR). Anti-human Fc antibody from the Human Antibody Capture Kit was immobilized on a CMS sensor chip to approximately 5000 RU using standard N-Hydroxysuccinimide/1-ethyl-3-(−3-dimethylaminopropyl) carbodiimide hydrochloride (NHS/EDC) coupling methodology. In separate analyses, the S1-Fc protein and RBD-Fc protein (1 ug/mL in a running buffer of 0.01 M HEPES pH 7.4, 0.15 M NaCl, 3 mM EDTA, 0.05% v/v Surfactant P20, HBS-EP+) were captured for 60 s at a flow rate 10 μL/min. Recombinant sACE2(R273K)-6his (SEQ ID NO:12) and sACE2 (“wild type” sequence)-6his (SEQ ID NO:5) were serially diluted in HBS-EP+. All measurements were conducted in HBS-EP+ buffer with a flow rate of 30 μL/minute. All BIACORE assays were performed at 25° C. A 1:1 (Langmuir) binding model was used to fit the data.

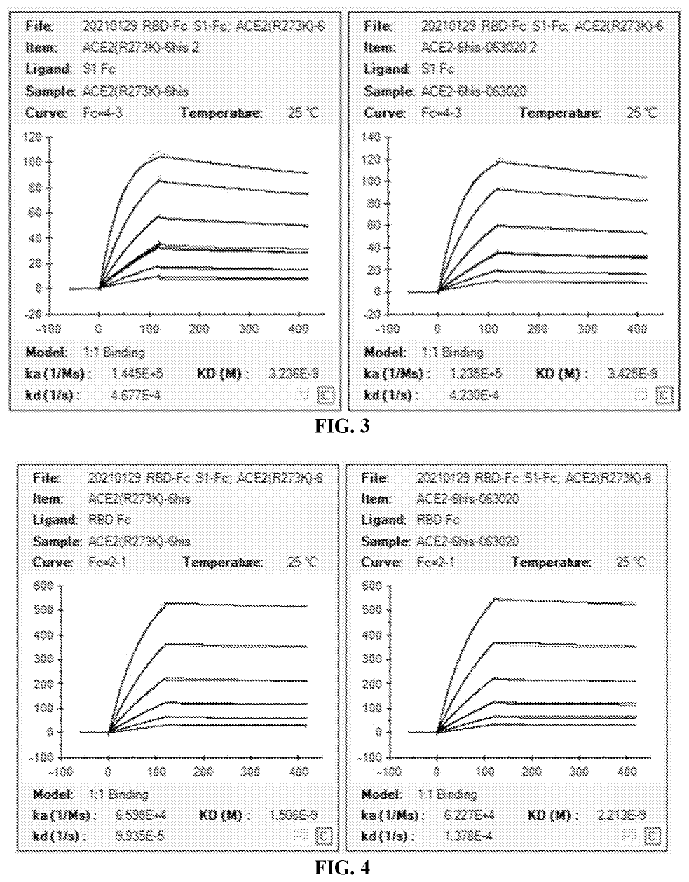

The SPR sensorgrams of sACE2(R273K)-6his and sACE2-6his binding to the SARS-CoV2 S1 protein are shown in FIG. 3 and the SPR sensorgrams of sACE2(R273K)-6his and sACE2-6his binding to the RBD of the SARS-CoV2 spike protein RBD are shown in FIG. 4. As shown in Table 1, The binding affinity (K_{D}) for binding of the sACE2(R273K)-6his for the 51 protein was found to be 3.24 nM, very similar to the binding affinity of the sACE2-6his for the 51 protein, 3.43 nM.

TABLE 1
Binding of sACE2(R273K)-6his and sACE2-his6 to S1 protein and RBD.

| | | Ka | Kd | K_{D} | Rmax | Chi² |
|---|---|---|---|---|---|---|
| Ligand | Sample | (1/Ms) | (1/s) | (M) | (RU) | (RU²) |
| S1-Fc | sACE2 | 1.45E+05 | 4.68E−04 | 3.24E−09 | 110.5 | 1.06 |
| | (R273K)-6his | | | | | |
| S1-Fc | sACE2-6his | 1.23E+05 | 4.23E−04 | 3.43E−09 | 126.9 | 0.475 |
| RBD-Fc | sACE2 | 6.60E+04 | 9.94E−05 | 1.51E−09 | 668.6 | 11.2 |
| | (R273K)-6his | | | | | |
| RBD-Fc | sACE2-6his | 6.23E+04 | 1.38E−04 | 2.21E−09 | 707.1 | 16.1 |

### Example 3. Focus Reduction Neutralization Test sACE2(R273K)-6his

To test the sACE2(R273K)-6his protein for its ability to inhibit infection of ACE2-expressing cells, the Focus Reduction Neutralization Test (FRNT) (Vanderheiden et al. (2020) Current Protocols in Immunology 131: e116 doi: 10.1002/cpim.116) was employed. Vero cells, which have been demonstrated to express ACE2 (Ren et al. (2006) J. Gen. Virology 87:1691-1695), were used as the target cells in these assays, and sACE2-6his (lacking the R273K mutation) was also assayed for comparison.

Vero cells were seeded at 1.8×10³ cells in 200 μl culture medium (DMEM+10% FBS+Glutamine+PS) per well in 96 flat bottom well plates (cell concentration 2.75×10⁵/ml) and incubated overnight in 37° C., 5% CO₂. The next day, the sACE2(R273K)-6his and sACE2-6his recombinant proteins were serially diluted in separate 96 round bottom well plates using culture medium. The medium was discarded from the wells containing the Vero cells and 50 μl of the recombinant protein dilutions were transferred from the round bottom plates to the Vero cells in flat bottom well plates. The plates were incubated for 2 hours at 36° C., 5% CO₂. After incubation, 1000 PFU/50 μl of SARS-COV-2 was added to each well and incubated at 36° C., 5% CO₂ for 24 hours (total volume was 100 μl per well).

After the 24 hour incubation, staining was performed by discarding the cell supernatants, fixing the plates with 4% paraformaldehyde (PFA)/PBS for 30 minutes and then staining with 40 μl of antibody (1 μg/ml anti-SARS-CoV-2 spike protein) in PBS+3% BSA+0.3% Triton X. The cells were incubated for 2 hours at room temperature or overnight at 4° C., after which the plates were washed with PBS and 40 μl of secondary antibody (goat anti-human IgG (Fc Sp)-FITC conjugate (1:1000) in PBS+3% BSA+0.3% Triton X) was added to each well (in some cases DAPI (1 μg/ml) was added with the secondary antibody). The plates were then incubated at RT for 2 hours, then washed. Viral foci were counted using a florescence microscope.

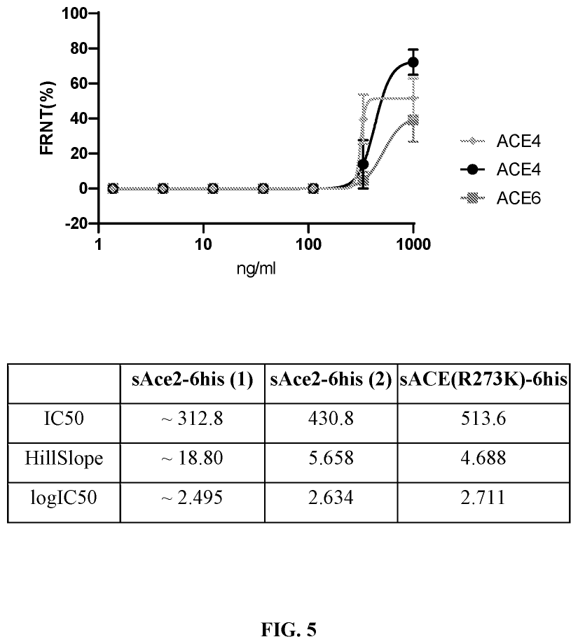

FIG. 5 provides the results of the neutralization assay, where independent samples of sACE2-his6 were found to have IC50s of approximately 313 ng/ml and 431 ng/ml, and the mutant sACE2(R273K)-6his was found to have an IC50 of approximately 514 ng/ml. All publications, patents and/or patent applications referenced herein are incorporated by reference in their entireties. To the extent any material incorporated by reference conflicts with the express content of this application, the express content of this application controls.

SEQUENCES
Protein
Homo sapiens
angiotensin-converting enzyme 2 (ACE2) precursor
NCBI Reference Sequence: NP_068576.1
Signal peptide is underlined
SEQ ID NO: 1
MSSSSWLLLSLVAVTAAQSTIEEQAKTFLDKFNHEAEDLFYQSSLASWNYNTNITEENVQNMNNAGDKWS
AFLKEQSTLAQMYPLQEIQNLTVKLQLQALQQNGSSVLSEDKSKRLNTILNTMSTIYSTGKVCNPDNPQE
CLLLEPGLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRFWTNLYS
LTVPFGQKPNIDVTDAMVDQAWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGFHEAVGEIMSLSAATPKHLKS
IGLLSPDFQEDNETEINFLLKQALTIVGTLPFTYMLEKWRWMVFKGEIPKDQWMKKWWEMKREIVGVVEP
VPHDETYCDPASLFHVSNDYSFIRYYTRTLYQFQFQEALCQAAKHEGPLHKCDISNSTEAGQKLFNMLRL
GKSEPWTLALENVVGAKNMNVRPLLNYFEPLFTWLKDQNKNSFVGWSTDWSPYADQSIKVRISLKSALGD
KAYEWNDNEMYLFRSSVAYAMRQYFLKVKNQMILFGEEDVRVANLKPRISFNFFVTAPKNVSDIIPRTEV
EKAIRMSRSRINDAFRLNDNSLEFLGIQPTLGPPNQPPVSIWLIVFGVVMGVIVVGIVILIFTGIRDRKK
KNKARSGENPYASIDISKGENNPGFQNTDDVQTSF
Homo sapiens
Soluble ACE2 precursor-“hrACE2” 726 amino acids, includes native
signal peptide (underlined), truncated at amino acid 726 of precursor
SEQ ID NO: 2
MSSSSWLLLSLVAVTAAQSTIEEQAKTFLDKFNHEAEDLFYQSSLASWNYNTNITEENVQNMNNAGDKWS
AFLKEQSTLAQMYPLQEIQNLTVKLQLQALQQNGSSVLSEDKSKRLNTILNTMSTIYSTGKVCNPDNPQE
CLLLEPGLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRFWTNLYS
LTVPFGQKPNIDVTDAMVDQAWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGFHEAVGEIMSLSAATPKHLKS
IGLLSPDFQEDNETEINFLLKQALTIVGTLPFTYMLEKWRWMVFKGEIPKDQWMKKWWEMKREIVGVVEP
VPHDETYCDPASLFHVSNDYSFIRYYTRTLYQFQFQEALCQAAKHEGPLHKCDISNSTEAGQKLFNMLRL
GKSEPWTLALENVVGAKNMNVRPLLNYFEPLFTWLKDQNKNSFVGWSTDWSPYADQSIKVRISLKSALGD
KAYEWNDNEMYLFRSSVAYAMRQYFLKVKNQMILFGEEDVRVANLKPRISFNFFVTAPKNVSDIIPRTEV
EKAIRMSRSRINDAFRLNDNSLEFLG
Protein
Artificial
ACE2 soluble ectodomain with heterologous (mouse IgG) 19 amino acid
N-terminal signal peptide and 6x his tag amino acids 18-726 of
precursor
SEQ ID NO: 3
MEWSWVFLFFLSVTTGVHSQSTIEEQAKTFLDKFNHEAEDLFYQSSLASWNYNTNITEENVQNMNNAGDK
WSAFLKEQSTLAQMYPLQEIQNLTVKLQLQALQQNGSSVLSEDKSKRLNTILNTMSTIYSTGKVCNPDNP
QECLLLEPGLNEIMANSLDYNERLWAWESWRSEVGKQLRPLYEEYVVLKNEMARANHYEDYGDYWRGDYE
VNGVDGYDYSRGQLIEDVEHTFEEIKPLYEHLHAYVRAKLMNAYPSYISPIGCLPAHLLGDMWGRFWTNL
YSLTVPFGQKPNIDVTDAMVDQAWDAQRIFKEAEKFFVSVGLPNMTQGFWENSMLTDPGNVQKAVCHPTA
WDLGKGDFRILMCTKVTMDDFLTAHHEMGHIQYDMAYAAQPFLLRNGANEGFHEAVGEIMSLSAATPKHL
KSIGLLSPDFQEDNETEINFLLKQALTIVGTLPFTYMLEKWRWMVFKGEIPKDQWMKKWWEMKREIVGVV
EPVPHDETYCDPASLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSPYADQSIKVRISLKSAL
GDKAYEWNDNEMYLFRSSVAYAMRQYFLKVKNQMILFGEEDVRVANLKPRISFNFFVTAPKNVSDIIPRT
EVEKAIRMSRSRINDAFRLNDNSLEFLGHHHHHH
Protein
Artificial
ACE 2 soluble ectodomain with heterologous (mouse IgG) 19 amino acid
N-terminal signal peptide (amino acids 18-732 of precursor), followed
by 6xHis
SEQ ID NO: 4
MEWSWVFLFFLSVTTGVHSQSTIEEQAKTFLDKFNHEAEDLFYQSSLASWNYNTNITEENVQNMNNAGDK
WSAFLKEQSTLAQMYPLQEIQNLTVKLQLQALQQNGSSVLSEDKSKRLNTILNTMSTIYSTGKVCNPDNP
QECLLLEPGLNEIMANSLDYNERLWAWESWRSEVGKQLRPLYEEYVVLKNEMARANHYEDYGDYWRGDYE
VNGVDGYDYSRGQLIEDVEHTFEEIKPLYEHLHAYVRAKLMNAYPSYISPIGCLPAHLLGDMWGRFWTNL
YSLTVPFGQKPNIDVTDAMVDQAWDAQRIFKEAEKFFVSVGLPNMTQGFWENSMLTDPGNVQKAVCHPTA
WDLGKGDFRILMCTKVTMDDFLTAHHEMGHIQYDMAYAAQPFLLRNGANEGFHEAVGEIMSLSAATPKHL
KSIGLLSPDFQEDNETEINFLLKQALTIVGTLPFTYMLEKWRWMVFKGEIPKDQWMKKWWEMKREIVGVV
EPVPHDETYCDPASLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSPYADQSIKVRISLKSAL
GDKAYEWNDNEMYLFRSSVAYAMRQYFLKVKNQMILFGEEDVRVANLKPRISFNFFVTAPKNVSDIIPRT
EVEKAIRMSRSRINDAFRLNDNSLEFLGIQPTLGHHHHHH
Protein
Artificial
ACE 2 soluble ectodomain (amino acids 18-732 of precursor) with 6xHis
tag
SEQ ID NO: 5
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGRFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYADQSIKVRISLKSALGDKAYEWNDNEMYLFRSSV
AYAMRQYFLKVKNQMILFGEEDVRVANLKPRISFNFFVTAPKNVSDIIPRTEVEKAIRMSRSRINDAFRL
NDNSLEFLGIQPTLGHHHHHH
Protein
Artificial
ACE2 soluble ectodomain (amino acids 18-740)
SEQ ID NO: 6
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGRFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYADQSIKVRISLKSALGDKAYEWNDNEMYLFRSSV
AYAMRQYFLKVKNQMILFGEEDVRVANLKPRISFNFFVTAPKNVSDIIPRTEVEKAIRMSRSRINDAFRL
NDNSLEFLGIQPTLGPPNQPPVS
Protein
Artificial
ACE2 soluble ectodomain (amino acids 18-732)
SEQ ID NO: 7
QSTIEEQAKTFLDKFNHEAEDLFYQSSLASWNYNTNITEENVQNMNNAGDKWS
AFLKEQSTLAQMYPLQEIQNLTVKLQLQALQQNGSSVLSEDKSKRLNTILNTMSTIYSTGKVCNPDNPQE
CLLLEPGLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRFWTNLYS
LTVPFGQKPNIDVTDAMVDQAWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGFHEAVGEIMSLSAATPKHLKS
IGLLSPDFQEDNETEINFLLKQALTIVGTLPFTYMLEKWRWMVFKGEIPKDQWMKKWWEMKREIVGVVEP
VPHDETYCDPASLEHVSNDYSFIRYYTRTLYgFQFQEALCQAAKHEGPLHKCDISNSTEAGQKLFNMLRL
GKSEPWTLALENVVGAKNMNVRPLLNYFEPLFTWLKDQNKNSFVGWSTDWSPYADQSIKVRISLKSALGD
KAYEWNDNEMYLFRSSVAYAMRQYFLKVKNQMILFGEEDVRVANLKPRISFNFFVTAPKNVSDIIPRTEV
EKAIRMSRSRINDAFRLNDNSLEFLGIQPTLG
Protein
Artificial
ACE2 soluble ectodomain (amino acids 18-726)
SEQ ID NO: 8
QSTIEEQAKTFLDKFNHEAEDLFYQSSLASWNYNTNITEENVQNMNNAGDKWS
AFLKEQSTLAQMYPLQEIQNLTVKLQLQALQQNGSSVLSEDKSKRLNTILNTMSTIYSTGKVCNPDNPQE
CLLLEPGLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRFWTNLYS
LTVPFGQKPNIDVTDAMVDQAWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGFHEAVGEIMSLSAATPKHLKS
IGLLSPDFQEDNETEINFLLKQALTIVGTLPFTYMLEKWRWMVFKGEIPKDQWMKKWWEMKREIVGVVEP
VPHDETYCDPASLFHVSNDYSFIRYYTRTLYQFQFQEALCQAAKHEGPLHKCDISNSTEAGQKLFNMLRL
GKSEPWTLALENVVGAKNMNVRPLLNYFEPLFTWLKDQNKNSFVGWSTDWSPYADQSIKVRISLKSALGD
KAYEWNDNEMYLFRSSVAYAMRQYFLKVKNQMILFGEEDVRVANLKPRISFNFFVTAPKNVSDIIPRTEV
EKAIRMSRSRINDAFRLNDNSLEFLG
Protein
Artificial
ACE2 soluble ectodomain (amino acids 18-708)
SEQ ID NO: 9
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGRFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYADQSIKVRISLKSALGDKAYEWNDNEMYLFRSSV
AYAMRQYFLKVKNQMILFGEEDVRVANLKPRISFNFFVTAPKNVSDIIPRTEVEKAIRMSR
Protein
Homo sapiens
ACE2 shortened extracellular domain (no signal peptide)-up to amino
acid 691 of precursor
SEQ ID NO: 10
STIEEQAKTFLDKFNHEAEDLFYQSSLASWNYNTNITEENVQNMNNAGDKWSAFLKEQSTLAQMYPLQEI
QNLTVKLQLQALQQNGSSVLSEDKSKRLNTILNTMSTIYSTGKVCNPDNPQECLLLEPGLNEIMANSLDY
NERLWAWESWRSEVGKQLRPLYEEYVVLKNEMARANHYEDYGDYWRGDYEVNGVDGYDYSRGQLIEDVEH
TFEEIKPLYEHLHAYVRAKLMNAYPSYISPIGCLPAHLLGDMWGRFWTNLYSLTVPFGQKPNIDVTDAMV
DQAWDAQRIFKEAEKFFVSVGLPNMTQGFWENSMLTDPGNVQKAVCHPTAWDLGKGDFRILMCTKVTMDD
FLTAHHEMGHIQYDMAYAAQPFLLRNGANEGFHEAVGEIMSLSAATPKHLKSIGLLSPDFQEDNETEINF
LLKQALTIVGTLPFTYMLEKWRWMVFKGEIPKDQWMKKWWEMKREIVGVVEPVPHDETYCDPASLFHVSN
DYSFIRYYTRTLYQFQFQEALCQAAKHEGPLHKCDISNSTEAGQKLFNMLRLGKSEPWTLALENVVGAKN
MNVRPLLNYFEPLFTWLKDQNKNSFVGWSTDWSPYADQSIKVRISLKSALGDKAYEWNDNEMYLFRSSVA
YAMRQYFLKVKNQMILFGEEDVRVANLKPRISFNFFVTAPKNV
Protein
Artificial
ACE2 minimal soluble ectodomain (amino acids 18-615)
SEQ ID NO: 11
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGRFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYAD
Protein
Artificial
ACE2 soluble ectodomain (amino acids 18-732), R273K mutation
SEQ ID NO: 12
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGKFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYADQSIKVRISLKSALGDKAYEWNDNEMYLFRSSV
AYAMRQYFLKVKNQMILFGEEDVRVANLKPRISFNFFVTAPKNVSDIIPRTEVEKAIRMSRSRINDAFRL
NDNSLEFLGIQPTLG
Protein
Artificial
ACE2 soluble ectodomain (amino acids 18-732), R273K mutation, includes
N-terminal heterologous signal peptide, C-terminal 6xhis tag
SEQ ID NO: 13
MEWSWVFLFFLSVTTGVHSQSTIEEQAKTFLDKFNHEAEDLFYQSSLASWNYNTNITEENVQNMNNAGDK
WSAFLKEQSTLAQMYPLQEIQNLTVKLQLQALQQNGSSVLSEDKSKRLNTILNTMSTIYSTGKVCNPDNP
QECLLLEPGLNEIMANSLDYNERLWAWESWRSEVGKQLRPLYEEYVVLKNEMARANHYEDYGDYWRGDYE
VNGVDGYDYSRGQLIEDVEHTFEEIKPLYEHLHAYVRAKLMNAYPSYISPIGCLPAHLLGDMWGKFWTNL
YSLTVPFGQKPNIDVTDAMVDQAWDAQRIFKEAEKFFVSVGLPNMTQGFWENSMLTDPGNVQKAVCHPTA
WDLGKGDFRILMCTKVTMDDFLTAHHEMGHIQYDMAYAAQPFLLRNGANEGFHEAVGEIMSLSAATPKHL
KSIGLLSPDFQEDNETEINFLLKQALTIVGTLPFTYMLEKWRWMVFKGEIPKDQWMKKWWEMKREIVGVV
EPVPHDETYCDPASLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSPYADQSIKVRISLKSAL
GDKAYEWNDNEMYLFRSSVAYAMRQYFLKVKNQMILFGEEDVRVANLKPRISFNFFVTAPKNVSDIIPRT
EVEKAIRMSRSRINDAFRLNDNSLEFLGIQPTLGHHHHHH
Protein
Artificial
ACE2 soluble ectodomain (amino acids 18-732), R273K mutation, includes
C-terminal 6xhis tag
SEQ ID NO: 14
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGKFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYADQSIKVRISLKSALGDKAYEWNDNEMYLFRSSV
AYAMRQYFLKVKNQMILFGEEDVRVANLKPRISFNFFVTAPKNVSDIIPRTEVEKAIRMSRSRINDAFRL
NDNSLEFLGIQPTLGHHHHHH
Protein
Artificial
ACE2 soluble ectodomain (amino acids 18-726), R273K mutation
SEQ ID NO: 15
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGKFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYADQSIKVRISLKSALGDKAYEWNDNEMYLFRSSV
AYAMRQYFLKVKNQMILFGEEDVRVANLKPRISFNFEVTAPKNVSDIIPRTEVEKAIRMSRSRINDAFRL
NDNSLEFLG
Protein
Artificial
ACE2 soluble ectodomain (amino acids 18-726), R273K mutation, includes
N-terminal heterologous signal peptide, C-terminal 6xhis tag
SEQ ID NO: 16
MEWSWVFLFFLSVTTGVHSQSTIEEQAKTFLDKFNHEAEDLFYQSSLASWNYNTNITEENVQNMNNAGDK
WSAFLKEQSTLAQMYPLQEIQNLTVKLQLQALQQNGSSVLSEDKSKRLNTILNTMSTIYSTGKVCNPDNP
QECLLLEPGLNEIMANSLDYNERLWAWESWRSEVGKQLRPLYEEYVVLKNEMARANHYEDYGDYWRGDYE
VNGVDGYDYSRGQLIEDVEHTFEEIKPLYEHLHAYVRAKLMNAYPSYISPIGCLPAHLLGDMWGKFWTNL
YSLTVPFGQKPNIDVTDAMVDQAWDAQRIFKEAEKFFVSVGLPNMTQGFWENSMLTDPGNVQKAVCHPTA
WDLGKGDFRILMCTKVTMDDFLTAHHEMGHIQYDMAYAAQPFLLRNGANEGFHEAVGEIMSLSAATPKHL
KSIGLLSPDFQEDNETEINFLLKQALTIVGTLPFTYMLEKWRWMVFKGEIPKDQWMKKWWEMKREIVGVV
EPVPHDETYCDPASLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSPYADQSIKVRISLKSAL
GDKAYEWNDNEMYLFRSSVAYAMRQYFLKVKNQMILFGEEDVRVANLKPRISFNFFVTAPKNVSDIIPRT
EVEKAIRMSRSRINDAFRLNDNSLEFLGHHHHHH
Protein
Artificial
ACE2 soluble ectodomain (amino acids 18-726), R273K mutation, includes
C-terminal 6xhis tag
SEQ ID NO: 17
QSTIEEQAKTFLDKFNHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGKFWTNLYSLTVPFGQKPNIDVTDAM
VDQAWDAQRIFKEAEKFFVSVGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFNMLRLGKSEPWTLALENVVGAK
NMNVRPLLNYFEPLFTWLKDQNKNSFVGWSTDWSPYADQSIKVRISLKSALGDKAYEWNDNEMYLFRSSV
AYAMRQYFLKVKNQMILFGEEDVRVANLKPRISFNFFVTAPKNVSDIIPRTEVEKAIRMSRSRINDAFRL
NDNSLEFLGHHHHHH
DNA
Artificial
Encodes precursor of sACE2(R273K)6xhis (SEQ ID NO: 13)
SEQ ID NO: 18
ATGGAGTGGAGCTGGGTTTTCCTGTTTTTTCTGTCCGTAACCACAGGGGTGCATAGTCAGTCCACTATCG
AGGAGCAAGCTAAAACTTTCCTGGACAAGTTTAATCATGAGGCCGAGGACCTGTTTTACCAGTCCTCACT
TGCTTCCTGGAATTATAATACTAATATCACTGAGGAAAACGTGCAAAATATGAACAACGCTGGCGATAAA
TGGTCTGCTTTCCTGAAAGAGCAGTCAACCCTTGCTCAGATGTATCCACTTCAGGAAATTCAGAATCTGA
CTGTTAAACTGCAGTTGCAGGCCTTGCAGCAGAACGGATCTAGTGTTCTTTCTGAGGATAAGTCTAAGAG
GCTGAATACCATCCTGAATACAATGTCAACCATCTACAGTACCGGAAAAGTGTGTAACCCAGACAATCCT
CAGGAATGCCTCCTGTTGGAGCCAGGTCTTAATGAGATTATGGCCAATAGCCTTGACTATAACGAACGTC
TGTGGGCATGGGAGTCATGGCGGTCTGAAGTTGGTAAACAGCTCCGACCACTGTACGAGGAGTACGTTGT
GCTGAAAAATGAGATGGCCAGGGCCAATCATTATGAAGACTACGGAGATTACTGGAGAGGAGATTACGAA
GTCAACGGGGTCGACGGTTACGATTACAGTCGGGGTCAACTGATCGAAGACGTGGAGCATACCTTTGAGG
AGATAAAGCCACTGTATGAGCATCTTCACGCATACGTGCGAGCTAAGCTGATGAATGCATATCCAAGTTA
CATTTCCCCTATCGGTTGTTTGCCTGCACATCTGCTCGGTGATATGTGGGGGAAGTTCTGGACCAATCTG
TATTCCCTGACTGTGCCCTTCGGCCAGAAACCAAACATCGATGTTACAGATGCTATGGTAGATCAAGCCT
GGGATGCTCAGAGGATTTTTAAGGAGGCTGAGAAATTCTTCGTTAGTGTGGGCCTCCCTAACATGACCCA
GGGTTTTTGGGAGAACAGCATGCTCACTGACCCCGGCAATGTCCAGAAGGCCGTTTGTCATCCAACCGCC
TGGGACTTGGGTAAGGGCGACTTCCGCATACTGATGTGTACCAAAGTCACTATGGATGACTTCCTCACTG
CTCACCATGAAATGGGACACATTCAGTATGACATGGCCTACGCCGCCCAGCCTTTTTTGCTTCGCAATGG
TGCCAATGAGGGTTTTCACGAGGCCGTAGGAGAGATCATGAGCCTGTCTGCCGCCACCCCAAAACATCTC
AAAAGTATCGGCCTGCTGTCTCCAGATTTCCAGGAAGATAATGAAACCGAGATTAACTTCCTGCTCAAAC
AAGCCCTTACAATCGTGGGTACCTTGCCCTTCACATATATGTTGGAAAAATGGAGGTGGATGGTGTTCAA
AGGTGAAATCCCTAAGGACCAGTGGATGAAGAAGTGGTGGGAAATGAAGCGCGAAATTGTTGGAGTCGTG
GAGCCAGTGCCTCACGACGAAACTTATTGCGATCCCGCCTCTCTGTTTCATGTGAGCAACGACTACAGCT
TCATTCGCTACTACACACGGACTCTCTATCAGTTTCAGTTTCAGGAGGCCCTGTGCCAAGCTGCTAAACA
CGAAGGACCTCTGCACAAATGTGATATTAGCAACTCCACCGAGGCTGGTCAAAAGCTCTTTAATATGCTC
CGTCTGGGAAAATCTGAACCTTGGACTCTGGCACTTGAAAATGTCGTGGGGGCAAAGAATATGAATGTCC
GTCCACTCCTCAACTACTTCGAGCCCCTCTTCACCTGGCTGAAGGATCAGAACAAGAACTCTTTCGTCGG
ATGGTCAACAGACTGGTCACCCTATGCTGATCAGTCAATTAAGGTGCGGATCTCCCTCAAAAGCGCACTG
GGCGATAAGGCTTACGAGTGGAACGATAATGAGATGTACTTGTTTAGGTCTTCCGTGGCCTATGCTATGA
GGCAATATTTCCTGAAAGTGAAAAATCAGATGATCCTGTTTGGAGAGGAGGACGTCAGGGTGGCAAATCT
GAAGCCTAGAATATCATTTAACTTCTTTGTTACTGCACCTAAGAACGTATCTGATATTATTCCCAGAACA
GAGGTGGAGAAGGCAATTCGGATGAGTAGAAGTCGGATTAACGATGCTTTCAGACTGAACGACAATTCTC
TGGAGTTCCTTGGAATCCAACCTACACTGGGCCACCATCACCACCATCATtga
Homo sapiens
Native ACE2 signal peptide
17 amino acids
SEQ ID NO: 19
MSSSSWLLLSLVAVTAA
Mus musculus
signal peptide, IgG heavy chain
19 amino acids
SEQ ID NO: 20
MEWSWVFLFFLSVTTGVHS
Homo sapiens
Interleukin 6 signal peptide
SEQ ID NO: 21
MNSFSTSAFGPVAFSLGLLLVLPAAFPAP
Artificial
6x his tag
SEQ ID NO: 22
HHHHHH
Artificial
8x his tag
SEQ ID NO: 23
HHHHHHHH
Artificial
10x his tag
SEQ ID NO: 24
HHHHHHHHHH

We claim:

1. A method of treating or preventing coronavirus infection comprising delivering a soluble ACE2 (sACE2) polypeptide to the lungs of a subject infected with or at risk of becoming infected with a coronavirus.

2. The method of claim 1, wherein the sACE2 polypeptide is administered by inhalation.

3. The method of claim 2, wherein the sACE2 polypeptide is formulated as a dry powder pharmaceutical composition.

4. The method of claim 2, wherein the sACE2 polypeptide is formulated as a liquid pharmaceutical composition.

5. The method of claim 1, wherein the administering uses a delivery device selected from the group consisting of a nebulizer, a metered dose inhaler, or a dry powder inhaler.

6. The method of claim 1, wherein the composition is administered more than once.

7. The method of claim 1, wherein the coronavirus is hCov-NL63, SARS-CoV, or SARS-CoV-2.

8. The method of claim 5, wherein the coronavirus is SARS-CoV-2.

9. A method according to claim 1, wherein the sACE2 polypeptide comprises an amino acid sequence having at least 95% identity to the amino acid sequence of any of SEQ ID NOs:5-12, 14, 15, and 17.

10. A method according to claim 9, wherein the sACE2 polypeptide comprises an amino acid sequence of any of SEQ ID NOs:5-12, 14, 15, and 17.

11. A method according to claim 9, wherein the sACE2 polypeptide comprises a his tag.

12. A method according to claim 9, wherein the sACE2 polypeptide comprises at least one mutation that decreases peptidase activity of the soluble ACE2 polypeptide.

13. A method according to claim 12, wherein at least one mutation is a mutation at an amino acid position corresponding the amino acid position of the human ACE2 precursor (SEQ ID NO:1) selected from the group consisting of R273, H345, H374, H378, and H505.

14. A method according to claim 13, wherein at least one mutation is selected from the group consisting of R273K, H345A, H345L, H374N, H378N, H505A, and H505L.

15. A method according to claim 14, wherein at least one mutation is R273K.

16. A method according to claim 15, wherein the sACE2 polypeptide comprises the amino acid sequence of SEQ ID NO:12, SEQ ID NO:14, SEQ ID NO:15, or SEQ ID NO:17.

17. A soluble mutant ACE2 polypeptide having reduced peptidase activity comprising an amino acid sequence having at least 95% identity to SEQ ID NO:12, SEQ ID NO:14, SEQ ID NO:15, or SEQ ID NO:17.

18. A soluble mutant ACE2 polypeptide according to claim 17, comprising an amino acid sequence having at least 95% identity to SEQ ID NO:12.

19. A soluble mutant ACE2 polypeptide according to claim 17, comprising the amino acid sequence of SEQ ID NO:12.

20. The soluble mutant ACE2 polypeptide of claim 17, further comprising a his tag.

21. The soluble mutant ACE2 polypeptide of claim 17, wherein coronavirus is SARS-CoV-2.

22. The soluble mutant ACE2 polypeptide of claim 17, wherein the soluble mutant ACE2 polypeptide inhibits infection of ACE2-expressing cells by SARS-CoV-2.

23. The soluble mutant ACE2 polypeptide of claim 22, wherein the soluble ACE2 polypeptide inhibits infection of ACE2-expressing cells by SARS-CoV-2 with an IC50 of between about 200 ng/ml and about 1 μg/ml.

24. The soluble mutant ACE2 polypeptide of claim 23, wherein the soluble mutant ACE2 polypeptide inhibits infection of ACE2-expressing cells by SARS-CoV-2 with an IC50 of between about 300 ng/ml and about 700 ng/ml.

25. A pharmaceutical formulation for pulmonary delivery comprising a soluble mutant polypeptide of claim 18 and a pharmaceutically acceptable excipient or carrier.

26. The pharmaceutical formulation of claim 25, wherein the soluble mutant ACE2 polypeptide comprises a his tag.

27. The pharmaceutical formulation of claim 26, wherein the soluble mutant ACE2 polypeptide comprises the amino acid sequence of SEQ ID NO:14.

28. The pharmaceutical formulation of any of claims 25-27, wherein the formulation is a dry powder formulation.

29. The formulation of any of claims 25-27, wherein the formulation is a liquid formulation.

30. A composition comprising a sACE2 protein formulated for pulmonary delivery for use in a method of treating or preventing infection by a coronavirus, wherein the method comprises delivering the sACE2 polypeptide of any of claims 17-24 or a pharmaceutical formulation of any of claims 25-27 to a subject infected with or at risk of becoming infected with a coronavirus.

31. A pharmaceutical formulation comprising a sACE2 protein formulated for pulmonary delivery for use in a method of treating or preventing infection by a coronavirus according to claim 30, wherein the formulation is a dry powder formulation.

32. A pharmaceutical formulation comprising a sACE2 protein formulated for pulmonary delivery for use in a method of treating or preventing infection by a coronavirus according to claim 30, wherein the formulation is a liquid formulation.

33. A nucleic acid molecule encoding a precursor polypeptide for producing a soluble mutant ACE2 polypeptide of claim 26.

34. The nucleic acid molecule of claim 33, comprising SEQ ID NO:18.

35. A nucleic acid molecule according to claim 33, wherein the nucleic acid molecule is an expression vector.
